SEQUENCE LISTING (8 sequences)

SEQ ID NO: 1 - DNA, 5148 bp, Rattus norvegicus
  CDS (1)..(5148)
atgtcagttcttatatcacagagtgtgataaattatgtggaagaagagaatattcctgctctgaaagctcttcttgagaagtgcaaagatgtagatgagaggaacgagtgtggccagacgccgctgatgctggctgcggagcagggcaatgtggaaatagtgaaagaactgttaaagaatggagccaactgcaatctggaagatctggataactggactgcccttatatcggcatctaaggaggggcacatccacattgtggaggagctgctgaagagcggggccagtctggagcaccgagacatgggtggatggacagctctcatgtgggcgtgctacaaaggcaggaccgacgtggtcgagctgcttctttctcatggtgccaacccaagtgtcactggtttgcagtatagtgtctacccaatcatctgggcagcgggcagaggccatgccgacatagtgcatcttctactgcagaatggtgcgaaagtcaattgctccgacaagtacggaacaacccccttggtatgggctgcccgaaagggtcatttggaatgtgtgaagcacttgttggccatgggagctgatgttgatcaagaaggagccaattcgatgactgcacttatcgtggcagtgaaaggaggctacacacagtccgtgaaagagattttgaaaaggaatccaaatgtgaacctaactgacaaggatggaaacacagctctgatgatcgcatcgaaggagggacatattgagattgtgcaggacctgctggacgccggaacatacgtgaacatacctgacaggagtggggatactgtattgatcggcgctgtgagaggtggtcatgttgaaattgttcgagcacttctccaaaaatatgctgatatagacattagagggcaggacaataagaccgcgctgtattgggccgttgaaaagggaaatgcgacaatggtgagggatatcctgcagtgcaatccggacacggaaatatgcacgaaggatggtgagacaccactgataaaagccaccaagatgagaaatattgaagtggtggagctgctgctggataagggggctaaggtgtctgctgtggacaagaaaggggacactcccctgcacgttgccatccgagggaggagccggagactggccgaacttcttttacggaaccccaaagatggacggttactctacagacccaacaaagcaggcgagactccctacaacattgactgcagccaccagaaaagcattttaactcaaatatttggagccagacacttgtctcctacggaaacagatggcgacatgcttggttatgatttgtatagcagtgccctggccgatattctcagtgaacccaccatgcaaccacccatttgtgtgggcttgtatgcacagtggggaagtgggaagtctttcttactcaagaaactagaagatgagatgaagacgtttgcaggacagcagaccgagcccctcttccagttctcgtggctcatagtgttcctcaccctgctgctgtgtggcgggctcggcctagtgtttgctttcccggttgatacaaacctcgccatagccatttcattgagtttcctggctctcatatatatattcttcatcgtcatttacttcggtggacggcgggaaggagagagttggaactgggcctgggctctcagtaccagactggcaagacacattgggtacctggagctcctcttcaaactgatgtttgtgaacccacctgaactgccggagcaaactacaaaggctttacctgtgaggtttttgtttacagattacaatagactctccagtgtgggaggagaaacttccttggctgaaatgatcgcgaccctctcagatgcttgtgagagagaattcggctttttggctacccgtctttttcgagtcttcaggactgaagagtctcagggtaaaaagaaatggaaaaaaacatgctgcctcccctcttttgtcatctttctttttattgttggctgcattattgctgggatcacccttctggctatattcagagttgacccgaaacatctgacagtgaatgctatcctcatttctatcgcatctgtcgtggggttggccttcgtgctcaattgtcgaacatggtggcaagtgctggactctctcctgaattctcagagaaaacgcctccatagtgctgcctccaaattacacaagttaaaaagtgaaggattcatgaaagtcctgaagtgtgaggtggagctgatggccaggatggcaaaaaccattgacagcttcactcaaaaccagacaaggctggtggttatcattgacggcctggatgcctgcgagcaggataaagtccttcagatgctggacactgtccgagttctgttttcaaagggcccttttattgccatttttgcaagtgatccgcatattatcataaaggccatcaaccagaacctgaatagtgtgcttcgtgactcaaatataaacggacatgactacatgcgcaatatagttcacttaccagtttttcttaatagtcgtgggcttagcaatgcaaggaaattccttgtaacttcagcaacaaatggggacattacatgctcagacaccacaggaacacaggaggacactgacagaagagtttcacagaacagccttggggagatgacaaagcttgggagcaaaacggctctcaacagaagggacacttaccgcagaagacagatgcagagaaccatcacgaggcagatgtcctttgatctcacaaagctgctggtcactgaggattggttcagtgacatcagccctcagaccatgagacggctactcaatattgtttctgtgacagggcggctactaagagccaatcagattactttcaactgggacaggctagccagctggatcaacctcaccgagcagtggccgtaccggacgtcttggcttatactatatctggaagagactgaaggtctccctgatcagatgaccttaaagaccatgtatgagagaatatcgaagaatatcccaacaactaaagatgttgagccgcttcttgaaattgatggagatataagaaactttgaagtcttcttgtcttcaaggactccagttctcgtggctcgagatgtaaagacctttttgccgtgcacagtaaacctggatcccaaacttcgggagatcatcgcagatgtccgagctgcaagagagcagatcaacataggaggcctggcctatcccccgctccctctgcacgaaggccctcctcggccaccttctgggtacagtcagcctgcatctgtctgctcttcagcatctttcaacgggccgttcccaggcggggttgtgtctccacagccccacagcagttactacagcggcttgtccggaccccagcaccccttctacaacagggcagccgtccctgccacaggcagctcactgttactgagttcaatgaccgtggatgtcgtatgtgagaaactgagacagatagaagggctggaccagaacatgatgcctcagtactgcacaacaatcaaaaaggcgaacataaatggccgggtattgtctcagtgtaacattgatgagctgaagaaagagatggctatgaactttggagactggcatctcttcagaagcatggtcttagaaatgagaagtgtggaaagccaggtggtccctgaagaccctcgttttctcaatgaaaactccagtgccccagttcctcatggagagtctgctcgccgctcttctcacactgagctgcccctcacggagctttccagccagactccatacaccctgaacttcagctttgaagaactgaacacgcttggccttgatgagggagcccctaggcacagtaacctgagttggcagtcacaaactcgcagaaccccaagtctctcgagtctcaattcccaggactccagtattgaaatttcaaagcttactgataaggtgcaggccgagtatagagacgcctatagagagtacattgctcagatgtcccagttagaagggggcacagggtcgtccacaataagcggcagatcttctccacatagcacatactacataggccagagctcttcagggggctccatccattctactctagaacaggaaagggggaaggagggtgagctaaagcaggaggatgggcggaagtcatttttaatgaagaggggggatgtcatagattactcctcatcaggggtgtccactaatgaggcctcacccctggaccccattactgaagaagatgagaaatccgaccagtcaggtagtaagctactcccaggcaagaaatcctcagaacggcccagtctcttccagacagacttgaagcttaaggggggtggtcttcgctaccagaaactgcccagtgatgaagatgagtcggggacaggaagagttcagataacaccccactgctcaaagatgataaggacaaaaaggctgaaggcaaagcagagagagtgtgcaagtccccaggagcacagtgccgagcccatcagaacgttcattaaagcaaaagagtatttgtccgatgccctccttgacaaaaaggattcctcagattcgggagtgaggtccaatgagagttctcccaaccactctctccacaatgaggcagcagacgactcccaactggaaaaggcaaacctcatagagcttgaagatgaaggccacagtgggaagcgggggatgccacacagtctgagtggcttgcaagatccaattatagctcggatgtccatttgctcggaagacaagaaaagcccttccgagtgcagcctgattgctagcagccctgaagaaagctggcctgcgtgccagaaggcctacaatctaaaccgaacgcccagcactgtgactctgaacaacaacactgcacccaccaacagagccaatcagaacttcgatgagatagagggaatcagggagacgtcccaggtcattctgaggcctggccccagtcccaacccaactgctgtgcagaatgaaaacctgaagagcatggcgcacaagcgaagccagcgctccagttacacaaggctctcgaaagatgcatctgagctacatgcagcctcctcggagagcacaggctttggagaagagagagagagcattctctga

SEQ ID NO: 2 - protein, 1715 aa, Rattus norvegicus
MSVLISQSVINYVEEENIPALKALLEKCKDVDERNECGQTPLMLAAEQGNVEIVKELLKNGANCNLEDLDNWTALISASKEGHIHIVEELLKSGASLEHRDMGGWTALMWACYKGRTDVVELLLSHGANPSVTGLQYSVYPIIWAAGRGHADIVHLLLQNGAKVNCSDKYGTTPLVWAARKGHLECVKHLLAMGADVDQEGANSMTALIVAVKGGYTQSVKEILKRNPNVNLTDKDGNTALMIASKEGHIEIVQDLLDAGTYVNIPDRSGDTVLIGAVRGGHVEIVRALLQKYADIDIRGQDNKTALYWAVEKGNATMVRDILQCNPDTEICTKDGETPLIKATKMRNIEVVELLLDKGAKVSAVDKKGDTPLHVAIRGRSRRLAELLLRNPKDGRLLYRPNKAGETPYNIDCSHQKSILTQIFGARHLSPTETDGDMLGYDLYSSALADILSEPTMQPPICVGLYAQWGSGKSFLLKKLEDEMKTFAGQQTEPLFQFSWLIVFLTLLLCGGLGLVFAFPVDTNLAIAISLSFLALIYIFFIVIYFGGRREGESWNWAWALSTRLARHIGYLELLFKLMFVNPPELPEQTTKALPVRFLFTDYNRLSSVGGETSLAEMIATLSDACEREFGFLATRLFRVFRTEESQGKKKWKKTCCLPSFVIFLFIVGCIIAGITLLAIFRVDPKHLTVNAILISIASVVGLAFVLNCRTWWQVLDSLLNSQRKRLHSAASKLHKLKSEGFMKVLKCEVELMARMAKTIDSFTQNQTRLVVIIDGLDACEQDKVLQMLDTVRVLFSKGPFIAIFASDPHIIIKAINQNLNSVLRDSNINGHDYMRNIVHLPVFLNSRGLSNARKFLVTSATNGDITCSDTTGTQEDTDRRVSQNSLGEMTKLGSKTALNRRDTYRRRQMQRTITRQMSFDLTKLLVTEDWFSDISPQTMRRLLNIVSVTGRLLRANQITFNWDRLASWINLTEQWPYRTSWLILYLEETEGLPDQMTLKTMYERISKNIPTTKDVEPLLEIDGDIRNFEVFLSSRTPVLVARDVKTFLPCTVNLDPKLREIIADVRAAREQINIGGLAYPPLPLHEGPPRPPSGYSQPASVCSSASFNGPFPGGVVSPQPHSSYYSGLSGPQHPFYNRAAVPATGSSLLLSSMTVDVVCEKLRQIEGLDQNMMPQYCTTIKKANINGRVLSQCNIDELKKEMAMNFGDWHLFRSMVLEMRSVESQVVPEDPRFLNENSSAPVPHGESARRSSHTELPLTELSSQTPYTLNFSFEELNTLGLDEGAPRHSNLSWQSQTRRTPSLSSLNSQDSSIEISKLTDKVQAEYRDAYREYIAQMSQLEGGTGSSTISGRSSPHSTYYIGQSSSGGSIHSTLEQERGKEGELKQEDGRKSFLMKRGDVIDYSSSGVSTNEASPLDPITEEDEKSDQSGSKLLPGKKSSERPSLFQTDLKLKGGGLRYQKLPSDEDESGTGRVQITPHCSKMIRTKRLKAKQRECASPQEHSAEPIRTFIKAKEYLSDALLDKKDSSDSGVRSNESSPNHSLHNEAADDSQLEKANLIELEDEGHSGKRGMPHSLSGLQDPIIARMSICSEDKKSPSECSLIASSPEESWPACQKAYNLNRTPSTVTLNNNTAPTNRANQNFDEIEGIRETSQVILRPGPSPNPTAVQNENLKSMAHKRSQRSSYTRLSKDASELHAASSESTGFGEERESIL

SEQ ID NO: 3 - DNA, 5305 bp, Homo sapiens
  CDS (158)..(5305)
ggaagaatatggccgccgggtgtggtgagggcgacgcgcttgcagtcgccgtctcttgcttccccgtcctctgacatcgcctgcagccgagcgggcccgttccgccggagctgaggaccaggtattcaaataaagttaattgcagctttctgtgaaaatgtcagttttgatatcacagagcgtcataaattatgtagaggaagaaaacattcctgctctgaaagctcttcttgaaaaatgcaaagatgtagatgagagaaatgagtgtggccagactccactgatgatagctgccgaacaaggcaatctggaaatagtgaaggaattaattaagaatggagctaactgcaatctggaagatttggataattggacagcacttatatctgcatcgaaagaagggcatgtgcacatcgtagaggaactactgaaatgtggggttaacttggagcaccgtgatatgggaggatggacagctcttatgtgggcatgttacaaaggccgtactgacgtagtagagttgcttctttctcatggtgccaatccaagtgtcactggtctgcagtacagtgtttacccaatcatttgggcagcagggagaggccatgcagatatagttcatcttttactgcaaaatggtgctaaagtcaactgctctgataagtatggaaccacccctttagtttgggctgcacgaaagggtcatttggaatgtgtgaaacatttattggccatgggagctgatgtggatcaagaaggagctaattcaatgactgcacttattgtggcagtgaaaggaggttacacacagtcagtaaaagaaattttgaagaggaatccaaatgtaaacttaacagataaagatggaaatacagctttgatgattgcatcaaaggagggacatacggagattgtgcaggatctgctcgacgctggaacatatgtgaacatacctgacaggagtggggatactgtgttgattggcgctgtcagaggtggtcatgttgaaattgttcgagcgcttctccaaaaatatgctgatatagacattagaggacaggataataaaactgctttgtattgggctgttgagaaaggaaatgcaacaatggtgagagatatcttacagtgcaatcctgacactgaaatatgcacaaaggatggtgaaacgccacttataaaggctaccaagatgagaaacattgaagtggtggagctgctgctagataaaggtgctaaagtgtctgctgtagataagaaaggagatactcccttgcatattgctattcgtggaaggagccggaaactggcagaactgcttttaagaaatcccaaagatgggcgattactttataggcccaacaaagcaggcgagactccttataatattgactgtagccatcagaagagtattttaactcaaatatttggagccagacacttgtctcctactgaaacagacggtgacatgcttggatatgatttatatagcagtgccctggcagatattctcagtgagcctaccatgcagccacccatttgtgtggggttatatgcacagtggggaagtgggaaatctttcttactcaagaaactagaagacgaaatgaaaaccttcgccggacaacagattgagcctctctttcagttctcatggctcatagtgtttcttaccctgctactttgtggagggcttggtttattgtttgccttcacggtccacccaaatcttggaatagcagtgtcactgagcttcttggctctcttatatatattctttattgtcatttactttggtggacgaagagaaggagagagttggaattgggcctgggtcctcagcactagattggcaagacatattggatatttggaactcctccttaaattgatgtttgtgaatccacctgagttgccagagcagactactaaagctttacctgtgaggtttttgtttacagattacaatagactgtccagtgtaggtggagaaacttctctggctgaaatgattgcaaccctctcggatgcttgtgaaagagagtttggctttttggcaaccaggctttttcgagtattcaagactgaagatactcagggtaaaaagaaatggaaaaaaacatgttgtctcccatcttttgtcatcttcctttttatcattggctgcattatatctggaattactcttctggctatatttagagttgacccaaagcatctgactgtaaatgctgtcctcatatcaatcgcatctgtagtgggattggcctttgtgttgaactgtcgtacatggtggcaagtgctggactcgctcctgaattcccaaagaaaacgcctccataatgcagcctccaaactgcacaaattgaaaagtgaaggattcatgaaagttcttaaatgtgaagtggaattgatggccaggatggcaaaaaccattgacagcttcactcagaatcagacaaggctggtggtcatcatcgatggattagatgcctgtgagcaggacaaagtccttcagatgctggacactgtccgagttctgttttcaaaaggcccgttcattgccatttttgcaagtgatccacatattatcataaaggcaattaaccagaacctcaatagtgtgcttcgggattcaaatataaatggccatgactacatgcgcaacatagtccacttgcctgtgttccttaatagtcgtggactaagcaatgcaagaaaatttctcgtaacttcagcaacaaatggagacgttccatgctcagatactacagggatacaggaagatgctgacagaagagtttcacagaacagccttggggagatgacaaaacttggtagcaagacagccctcaatagacgggacacttaccgaagaaggcagatgcagaggaccatcactcgccagatgtcctttgatcttacaaaactgctggttaccgaggactggttcagtgacatcagtccccagaccatgagaagattacttaatattgtttctgtgacaggacgattactgagagccaatcagattagtttcaactgggacaggcttgctagctggatcaaccttactgagcagtggccataccggacttcatggctcatattatatttggaagagactgaaggtattccagatcaaatgacattaaaaaccatctacgaaagaatatcaaagaatattccaacaactaaggatgttgagccacttcttgaaattgatggagatataagaaattttgaagtgtttttgtcttcaaggaccccagttcttgtggctcgagatgtaaaagtctttttgccatgcactgtaaacctagatcccaaactacgggaaattattgcagatgttcgtgctgccagagagcagatcagtattggaggactggcgtaccccccgctccctctacatgagggtcctcctagggcgccatcagggtacagccagcccccatccgtgtgctcttccacgtccttcaatgggcccttcgcaggtggagtggtgtcaccacagcctcacagcagctattacagcggcatgacgggccctcagcatcccttctacaacagggggtcaggcccagccccaggcccagtggtattactgaattcactgaatgtggatgcagtatgtgagaagctgaaacaaatagaagggctggaccagagtatgctgcctcagtattgtaccacgatcaaaaaggcaaacataaatggccgtgtgttagctcagtgtaacattgatgagctgaagaaagagatgaatatgaattttggagactggcaccttttcagaagcacagtactagaaatgagaaacgcagaaagccacgtggtccctgaagacccacgtttcctcagtgagagcagcagtggcccagccccgcacggtgagcctgctcgccgcgcttcccacaacgagctgcctcacaccgagctctccagccagacgccctacacactcaacttcagcttcgaagagctgaacacgcttggcctggatgaaggtgcccctcgtcacagtaatctaagttggcagtcacaaactcgcagaaccccaagtctttcgagtctcaattcccaggattccagtattgaaatttcaaagcttactgataaggtgcaggccgagtatagagatgcctatagagaatacattgctcagatgtcccagttagaagggggccccgggtctacaaccattagtggcagatcttctccacatagcacatattacatgggtcagagttcatcagggggctctattcattcaaacctagagcaagaaaaggggaaggatagtgaaccaaagcccgatgatgggaggaagtcctttctaatgaagaggggagatgttatcgattattcatcatcaggggtttccaccaacgatgcttcccccctggatcctatcactgaagaagatgaaaaatcagatcagtcaggcagtaagcttctcccaggcaagaaatcttccgaaaggtcaagcctcttccagacagatttgaagcttaagggaagtgggctgcgctatcaaaaactcccaagtgacgaggatgaatctggcacagaagaatcagataacactccactgctcaaagatgacaaagacagaaaagccgaagggaaagtagagagagtgccgaagtctccagaacacagtgctgagccgatcagaaccttcattaaagccaaagagtatttatcggatgcgctccttgacaaaaaggattcatcggattcaggagtgagatccagtgaaagttctcccaatcactctctgcacaatgaagtggcggatgactcccagcttgaaaaggcaaatctcatagagctggaagatgacagtcacagcggaaagcggggaatcccacatagcctgagtggcctgcaagatccaattatagctcggatgtccatttgttcagaagacaagaaaagcccttccgaatgcagcttgatagccagcagccctgaagaaaactggcctgcatgccagaaagcctacaacctgaaccgaactcccagcaccgtgactctgaacaacaatagtgctccagccaacagagccaatcaaaatttcgatgagatggagggaattagggagacttctcaagtcattttgaggcctagttccagtcccaacccaaccactattcagaatgagaatctaaaaagcatgacacataagcgaagccaacgttcaagttacacaaggctctccaaagatcctccggagctccatgcagcagcctcttctgagagcacaggctttggagaagaaagagaaagcattctttga

SEQ ID NO: 4 - protein, 1715 aa, Homo sapiens
MSVLISQSVINYVEEENIPALKALLEKCKDVDERNECGQTPLMIAAEQGNLEIVKELIKNGANCNLEDLDNWTALISASKEGHVHIVEELLKCGVNLEHRDMGGWTALMWACYKGRTDVVELLLSHGANPSVTGLQYSVYPIIWAAGRGHADIVHLLLQNGAKVNCSDKYGTTPLVWAARKGHLECVKHLLAMGADVDQEGANSMTALIVAVKGGYTQSVKEILKRNPNVNLTDKDGNTALMIASKEGHTEIVQDLLDAGTYVNIPDRSGDTVLIGAVRGGHVEIVRALLQKYADIDIRGQDNKTALYWAVEKGNATMVRDILQCNPDTEICTKDGETPLIKATKMRNIEVVELLLDKGAKVSAVDKKGDTPLHIAIRGRSRKLAELLLRNPKDGRLLYRPNKAGETPYNIDCSHQKSILTQIFGARHLSPTETDGDMLGYDLYSSALADILSEPTMQPPICVGLYAQWGSGKSFLLKKLEDEMKTFAGQQIEPLFQFSWLIVFLTLLLCGGLGLLFAFTVHPNLGIAVSLSFLALLYIFFIVIYFGGRREGESWNWAWVLSTRLARHIGYLELLLKLMFVNPPELPEQTTKALPVRFLFTDYNRLSSVGGETSLAEMIATLSDACEREFGFLATRLFRVFKTEDTQGKKKWKKTCCLPSFVIFLFIIGCIISGITLLAIFRVDPKHLTVNAVLISIASVVGLAFVLNCRTWWQVLDSLLNSQRKRLHNAASKLHKLKSEGFMKVLKCEVELMARMAKTIDSFTQNQTRLVVIIDGLDACEQDKVLQMLDTVRVLFSKGPFIAIFASDPHIIIKAINQNLNSVLRDSNINGHDYMRNIVHLPVFLNSRGLSNARKFLVTSATNGDVPCSDTTGIQEDADRRVSQNSLGEMTKLGSKTALNRRDTYRRRQMQRTITRQMSFDLTKLLVTEDWFSDISPQTMRRLLNIVSVTGRLLRANQISFNWDRLASWINLTEQWPYRTSWLILYLEETEGIPDQMTLKTIYERISKNIPTTKDVEPLLEIDGDIRNFEVFLSSRTPVLVARDVKVFLPCTVNLDPKLREIIADVRAAREQISIGGLAYPPLPLHEGPPRAPSGYSQPPSVCSSTSFNGPFAGGVVSPQPHSSYYSGMTGPQHPFYNRGSGPAPGPVVLLNSLNVDAVCEKLKQIEGLDQSMLPQYCTTIKKANINGRVLAQCNIDELKKEMNMNFGDWHLFRSTVLEMRNAESHVVPEDPRFLSESSSGPAPHGEPARRASHNELPHTELSSQTPYTLNFSFEELNTLGLDEGAPRHSNLSWQSQTRRTPSLSSLNSQDSSIEISKLTDKVQAEYRDAYREYIAQMSQLEGGPGSTTISGRSSPHSTYYMGQSSSGGSIHSNLEQEKGKDSEPKPDDGRKSFLMKRGDVIDYSSSGVSTNDASPLDPITEEDEKSDQSGSKLLPGKKSSERSSLFQTDLKLKGSGLRYQKLPSDEDESGTEESDNTPLLKDDKDRKAEGKVERVPKSPEHSAEPIRTFIKAKEYLSDALLDKKDSSDSGVRSSESSPNHSLHNEVADDSQLEKANLIELEDDSHSGKRGIPHSLSGLQDPIIARMSICSEDKKSPSECSLIASSPEENWPACQKAYNLNRTPSTVTLNNNSAPANRANQNFDEMEGIRETSQVILRPSSSPNPTTIQNENLKSMTHKRSQRSSYTRLSKDPPELHAAASSESTGFGEERESIL

SEQ ID NO: 5 - DNA, 4197 bp, Caenorhabditis Elegans
  CDS (1)..(4197)
atggttcaaacactgagaagaccgtggcaagaagcggcaagcagtgcttttgccgtcgccagtgctcttccggtgactatgaatagtacacaaattgctgagctcttcgaacaggttgagcatggcaccacagaactcagatgtgcactcaccgcagaaatatcagctctcagaaacgctaatggagaatcacttctcacagttgccgttcgatcaggaaacacagcagttgccaaacaattagcacaattggatccagacgcaattgatgaaactgacaatgaaggttggagtgctcttctgaatgcagctcactgtggtcatgttgacattgttcgattattaattgataatggagcatctgttgatcaacctgatttaatgggatggagtcctcttatgtgggcagtttataaaaatcatttagatgtggttgatctacttgttaacgcaaaagtaaatgcctgtgacaaattcggaagtactgcattaatttgggcagctcgaaaaggacatcttcctgttgtacaactacttttaaattctggagctgaggtggatgcagttggaatgtactcatccacagctttaatgttggcaacacgtggaaactttattcaagtagttgagcttcttctgacacgagaaccaaatgttaatgtagctgatcaaaatggtttgacagctctcggaatggcagctcgtgatggatatgctgatatatgtgaatctcttattaattcgggtgcttttgtgaatcagtgtgatagatttggaaactggattcttacgtcagcagttcgaagtggaaatgcggcaattgttagaatgattctcgacaaatttgctgatatcaattgtcaagactctgaaaaacggacaccacttcatctggccatcgataaatcattcaatgatatcgcctacattttattggagaaaaaaccgaatttggaactgaaaaataaggacggtgaaactccacttcttcgtgctgctaaatgtcgtcatgtacatctgtgtacctacttgatgagtttcggagctaaactcgctgcagttgataattgtggagataatgcacttcatttggcactgagagcacgtagtcgacgattaacacaggcacttttgtccaatccatcagattctcggttactgtaccgacccaataagcttggtcagacaccgtattccattgacttatcaaatcctcaacctattctgccactaatttttggaccaattgatgcggaagataaaatggatacagctatgggatatgatgtatattctaatgttttagctgatattgtgtgtgaaccttctctttctcttccacttacaattggactttacgccaaatggggatccggaaaatcagcacttctcgcaaaattgaaagaggcaatgcacagtttctcaagggattggcttgatggagtttcgctttcagtttcgtttgccttattctttgccatattcctcttttttggaatgttctcactaacttttacaatgctcattgccatttctaactcagtcacagcatatctcatctcatggtccgtctttcttctcattttcatcatattctgttcactgattgttgtggtttattatggggatcgcaagaactggtatacttcaatggatatagccaatttttttgcacgggtgttcagtcggatccgattggtttacaacattttaacacttcatgcaccgatgaacagtgaagatagtgcaagtatgcctgttagttttctctttgccgactaccaccgtctttcttctattggaggcgagcaagctcttgctaaaatcgtggcgacattgttcgaggcagctgaaactcattttggagtgcttccagttcgacttttctgttgcatgaaaccaccataccctggaattcatggatctttacgtcgtcactgcggtgttccccatgttattcttctgatcgttgccgtctttctactaatcatggctcaagtttttggaacagtttggctactttctgatcgagatccgaataacttcaatctattcatagcaattgcattcctttgtggatttgtaatgattgccatctatccattagcattaattataatgtattcatggacaaatgtaccaagacgaagagtcaatgcagctgctcggaatgctcataaattacgatttgaaggtctaatgcaaaagttacagaccgaggttgatcttcttgctgatatgatccgatcactcgatgctttcactagaagtcacacacgtctcgttgtggtggtcgatgggctcgataattgtgagcaggaacgaatggttcagactcttgatgctcttgagctattattctcggcccgcaagcatcgccccttcatcacaattattgctgtggatccacatgtaatagtatctgcaatcaatcataatatgcattcagcactgtctggcactgaactcactggacacgactatctgaaaaacatcatcagtatgccgttctatcttcataattctgctcttcgtcagctacaatctaaacttcgtgaaaaacgagaatcgatggcagaatggaaagaaaggttcaaacgtcaagacacattctatggatcacatctttcattgagagaagctgatggaagaacaagcagaaagaagtccactgttccaaacatgaactcgaatagtgtggttggtcggaatatgaatgacggaattcttggcgaagactacttttccaatatgaatcctcgtgccatgcgtcgtattgtcaatgctttaactctaactggacgtctcatgagagcttttgaaattgacttttcatggatgtcactcggacattgggtatctcttctcgaacaatggccttcacgaatgtgttggcttattgatagagctcttgaagttcacaacaatcagcttcttctatctgaagtatactatcaactcaaggatcacattccagctcaggatgatttgatgcagctcgacagaaatttggagaattttgaaggtttcctagattctaaaggaatcccatcagcagagagacttactgttggacatgtcaagaaatttgtcccctgtaccagcagtttggatccttatttgagaaagttgattcgcgagagaagtaaaggattagtggatattgaagcacaagttggaagtgcaggaatggctattccaccgaatgcgagacttttgtttagtgatgatttaacatggatgtcgattgatactccacttgtcgaaatgaagcttgatgcagttgtcaacctgatccgtaaaatagacattccatcaaatcgcctcgattcaattctcgatagattctatcaattaaacttatgtggattggttcttgccacgtgtccattgccagaactgaaggatagtatgcaattaccacttggtgattggactttgattcgtttgcttcttgaaactcttaaagtatttggctcctctccaccaggactccgagtggataaacgaaaagcactgacactccgcgaagaggatgaggaagaggaaattgaagaggcagcagaagctgctttgaactctgaaaaagagcgtgctccacttcttggatctgtgagagctgaacagagaagacggtccacaattgttcagaatgccactgagctgagcattgatcacaaatgtcttatggagaagttatcgggaatggatttgactgaaaccgaaggtgatgtcaatgaaatgcatttctctcatttttcgtcgtcaactgatggaccatctccaatggctgatggattccttccagcttcagtttctgctgctccatcagtcagattcgacgataatattaatgatctggaacgtgaagcttctgacgctgacagtacacaatcccgatacgattcgaaagaaaatcttcttgaagatgaaagaagtgcttctccaccggcacatgttgatttaatgagatttgacagtggaaatacaagcagtgatttcgtacggtccagttaccgtacctcatcgagatatcgtagaatgagccgaggagacttgatgcgaggaatgcaccagaatgacacaatccgaagcaaaataaattttcagaaccagcaaactgtacaacgacgtggtggagacgactttgagcgtatccaacttgaacaactattcaatcgacaatccgatgcttaa

SEQ ID NO: 6 - protein, 1398 aa, Caenorhabditis Elegans
MVQTLRRPWQEAASSAFAVASALPVTMNSTQIAELFEQVEHGTTELRCALTAEISALRNANGESLLTVAVRSGNTAVAKQLAQLDPDAIDETDNEGWSALLNAAHCGHVDIVRLLIDNGASVDQPDLMGWSPLMWAVYKNHLDVVDLLVNAKVNACDKFGSTALIWAARKGHLPVVQLLLNSGAEVDAVGMYSSTALMLATRGNFIQVVELLLTREPNVNVADQNGLTALGMAARDGYADICESLINSGAFVNQCDRFGNWILTSAVRSGNAAIVRMILDKFADINCQDSEKRTPLHLAIDKSFNDIAYILLEKKPNLELKNKDGETPLLRAAKCRHVHLCTYLMSFGAKLAAVDNCGDNALHLALRARSRRLTQALLSNPSDSRLLYRPNKLGQTPYSIDLSNPQPILPLIFGPIDAEDKMDTAMGYDVYSNVLADIVCEPSLSLPLTIGLYAKWGSGKSALLAKLKEAMHSFSRDWLDGVSLSVSFALFFAIFLFFGMFSLTFTMLIAISNSVTAYLISWSVFLLIFIIFCSLIVVVYYGDRKNWYTSMDIANFFARVFSRIRLVYNILTLHAPMNSEDSASMPVSFLFADYHRLSSIGGEQALAKIVATLFEAAETHFGVLPVRLFCCMKPPYPGIHGSLRRHCGVPHVILLIVAVFLLIMAQVFGTVWLLSDRDPNNFNLFIAIAFLCGFVMIAIYPLALIIMYSWTNVPRRRVNAAARNAHKLRFEGLMQKLQTEVDLLADMIRSLDAFTRSHTRLVVVVDGLDNCEQERMVQTLDALELLFSARKHRPFITIIAVDPHVIVSAINHNMHSALSGTELTGHDYLKNIISMPFYLHNSALRQLQSKLREKRESMAEWKERFKRQDTFYGSHLSLREADGRTSRKKSTVPNMNSNSVVGRNMNDGILGEDYFSNMNPRAMRRIVNALTLTGRLMRAFEIDFSWMSLGHWVSLLEQWPSRMCWLIDRALEVHNNQLLLSEVYYQLKDHIPAQDDLMQLDRNLENFEGFLDSKGIPSAERLTVGHVKKFVPCTSSLDPYLRKLIRERSKGLVDIEAQVGSAGMAIPPNARLLFSDDLTWMSIDTPLVEMKLDAVVNLIRKIDIPSNRLDSILDRFYQLNLCGLVLATCPLPELKDSMQLPLGDWTLIRLLLETLKVFGSSPPGLRVDKRKALTLREEDEEEEIEEAAEAALNSEKERAPLLGSVRAEQRRRSTIVQNATELSIDHKCLMEKLSGMDLTETEGDVNEMHFSHFSSSTDGPSPMADGFLPASVSAAPSVRFDDNINDLEREASDADSTQSRYDSKENLLEDERSASPPAHVDLMRFDSGNTSSDFVRSSYRTSSRYRRMSRGDLMRGMHQNDTIRSKINFQNQQTVQRRGGDDFERIQLEQLFNRQSDA

SEQ ID NO: 7 - DNA, 4497 bp, Drosophila
  CDS (1)..(4497)
atgttcaaatcccgcctaaagacgaatccgggtgggagtgagaacataaatcgcttcggagactccatgggttcgctgggacatcgtgccttgttgcagtacatcgataataatgatatatccggtctgcgcgccattttggacagccgacatcttaccatcgatgatcgggacgagaatgccaccacggtgttgatggtagttgctggacgaggcttaactgcctttgttcgcgagttcctggcccgcggtgccgatgttcaagcggaggatctggacaactggacggcactgctgtgtgcctcccgaaacgggcacttagatgttgtccaactgctgctggaccatggcgctgaagtggagcatcgcgatatgggtggctggaccagtttgatgtgggcagcttaccgtggacacacggagctggtgcgactgctgctggacaaaggagccgatggcaatgcccatggtaactaccacctgggcgcccttttgtgggcggcgggccgtggctacaaggacatcgtggagctgctggtgcaacgcggcgctaaagtgaatgtgggtgataagtacggaaccacagccctggtgtgggcatgccggcgcggaaacgttgagattgtggataccttacttaaggctggagccaatgtggacaccgctggcatgtattcgtggacgccactgctcgttgcggccgccggtggccacacagattgcgttagttccattctcgagaagaaaccaaatgtaaatgccttggacaaggatggaatgactgccctttgcatagcgagtcgagagggtttccaggatattgccgcctcccttattgcggctggtgcctacataaatatccaggatcgcggagcagacacgccgctcatccatgccgttaaagcaggacatcgaactgtagtggaggctttgctcaaaaaacatgccgatgtggatatacagggaaaggatcgcaagacggccatttacaccgcggtggagaaaggacatacgccgattgttaaactcctgttggccaccaatcccgacctcgaatccgccaccaaggatggcgacactccgctgctgagagctgttagaaaccgcaacttggagattgtgcacttattgctagatcgaaaggccaaggtgaccgctagcgacaaaaggggcgacacctgcctgcacatcgcaatgcgggcgaggagcaagacgattgtggaggcccttctgcgaaatcccaagcacagtcagcttctatatagagccaacaaagctggagaaacgccctacaacattgactccctgcaccagaagaccatattgggtcaagtgtttggcgccagacgtctgaacaccaacgaggactccgagggaatgctgggctacgaactgtactcctctgccttggcggatgtgctcagtgagccgacattaaccactcccattaccgttggactctacgccaaatggggcagtggaaagagtttcctgctaaataagctgcgcgacgagatgaacaactttgcgcgccaatgggcggaaccaccgattcgaaccagtggtctgctcttcatcgtctgtcttcatgtggcattgctcattggaactattgtgggactgagcacttggtcagcggttgtgggcgtgtcggcggccgtcggcttcttgctgctcgcctatctgctcctggcagctgttagatactgtaattaccaaatggacatgcagtgggcctactctgtgcaacacggtctggagaagagaatgacaagattgcgtttgatacttcaggtggccttttgccatcccccgggaccccaatcggattcacaggcaaagcccgttcggtttcactttgcggaggccaacagcgcttcgcccacaggtgatggagcagtggctcatatgctggccgctctcttagatgctatagaatctcactacggttggctggccacgcgattgtaccgagccttccgtccaaagtgcttaaaagtggatgttggctggcgttggcgtcgcatgtgctgtatcccaatcgttttgatcttcgaacttgctctcgtcaccgtggttactggcatctctctcaccgtggcctacttcacgtttgccgatgagaaggaaaaggaacatatattagtggctctctatgtaattgctgccgtgatgggcacgctgatctgtacgcatctccatgttctggccaaggtgtttgtatccctgttcacctcccacattcgagtactaaaaagggcagttcggtccagtgagtcggctccattgactatgctgggtgccgaggtggccgtgatgacggacatggtcaagtgcctggacgcgttcaccaatcagcagagccgcttagttggcgtgatcgatgccttggattcctgcgacacggagaggattctcacccttctaaatgcggtgcaaacgcttctttcttcacctaaccgaccatttgtcttgctcatttctgtggatccccatgtcatagccaaagcagcggaggctaatagtcgacgactctttacggagggcggaatcggaggacatgacttcctgagaaacttggtgcatctgcctgtttatctgcaaaactccggactgagaaaggtgcaacgagcccagatgacagcgctgctgttcaagcgaagtggcggaggagattaccagacagacgatggacccacattgggtcactctgtatccgctcgtcgactgtccaatgcctctgaaataatctccagtcaggagaagctgcgcggacccgcccgtggaggtggtggaaagaagctgcgtctctccgaatccgtggccagctcaactggttccaatctccatcgcctgggtcagaatccccagaccgtgctcgatctatcgcgcattgtgctcacggatgattacttcagcgatgtgaatccacgaagtatgcgtcgcctaatgaacgtgatctacatcacggtgcgcttgctcaaggccttccagattgagttcagctggtatcgcctgagttcgtggatcaatctgactgagcagtggcctttgagagcaagtatgatagtgctgcatcacgatcagttcatggacagcaatgcggatgagagtgtatcgctgcaaagcgtttacgagaaattgcgacccaaactcgcttacttgcgagaagctgctccacttctagagttggatagggatgaacgaaagctcgacgccttcctgcagctgcacaaatcagatttactcgtggcggatctccgcatcttcttgcccttcaccattaatctggatccctacttaagaaaggtcttaaaggaggatcagcaaaccatcgaggacgagggctccctagtgatacaagcaaggcccagcgtttccaatacaatgcgtcaattcccagcgcccaccacttacgtgccttcgccgcaggcttatccaccctaccaaatgttccagaacgagtatcctgccaatgagttgcgctccaggaatctcagcacgagcacagagcctgtcaccccgctcattaactcacctagtgattcgtttggtgatgatatcctgcaaaccaagctgaccgatttgaccgttgagggagtcatcagcctgctggatcggattgaggacatgaagccagcgttgcccaaactagcgcccgtgctccgcgagaatgcaatcaatggacgtgtgctgaagcactgcgatatgccggatctgaaatcggttctgggcctgagctttggccactgggagctgttccgcctgctgatcaccacgttgcgggaatgcgagcgattgccacggaagcaacagcgtcagcagcagcagccgggtgcgttggaggcaccatcgaatgtcccgatgattaaggatgtgacggatgccctgatgcagccacccagagagtccctctcgcgaaagaactccgtcagtcatatggaaaagcaggtcacgctggaggagcagatgatctgcggcaccttgcagactctgaatgaggaggcgtacgaggatgtggccagtagcgagcgaccgagtcccacaggtgagatgttggcagcagtcgcacaactgcaattagcacccatccgcgagtcttccgagttcggatcgccttccgacgatcagaagcagtatggggtcaagataagcaacaacaacaacaacaaccagtacttgcatgcggagtacaaccggagcgtcagctcgcattccctgcagagtctgagcactctggtgggtgcacccgttggtcatggtggttcaggtggcagtcacctacacctgggcaatggtaacgatctgagcgatagcacgctcgatctaatgcatgtagactcggtatttggtggtggtggtggaggtggttatcatcgggcatcgcgccagatctcgataagcagcgagctgctgcacgagtccagttccttgcccatggtggtggtcattcccaatgcctcgggcgaacaggcctacgacgacaccaagctctga

SEQ ID NO: 8 - protein, 1498 aa, Drosophila
MFKSRLKTNPGGSENINRFGDSMGSLGHRALLQYIDNNDISGLRAILDSRHLTIDDRDENATTVLMVVAGRGLTAFVREFLARGADVQAEDLDNWTALLCASRNGHLDVVQLLLDHGAEVEHRDMGGWTSLMWAAYRGHTELVRLLLDKGADGNAHGNYHLGALLWAAGRGYKDIVELLVQRGAKVNVGDKYGTTALVWACRRGNVEIVDTLLKAGANVDTAGMYSWTPLLVAAAGGHTDCVSSILEKKPNVNALDKDGMTALCIASREGFQDIAASLIAAGAYINIQDRGADTPLIHAVKAGHRTVVEALLKKHADVDIQGKDRKTAIYTAVEKGHTPIVKLLLATNPDLESATKDGDTPLLRAVRNRNLEIVHLLLDRKAKVTASDKRGDTCLHIAMRARSKTIVEALLRNPKHSQLLYRANKAGETPYNIDSLHQKTILGQVFGARRLNTNEDSEGMLGYELYSSALADVLSEPTLTTPITVGLYAKWGSGKSFLLNKLRDEMNNFARQWAEPPIRTSGLLFIVCLHVALLIGTIVGLSTWSAVVGVSAAVGFLLLAYLLLAAVRYCNYQMDMQWAYSVQHGLEKRMTRLRLILQVAFCHPPGPQSDSQAKPVRFHFAEANSASPTGDGAVAHMLAALLDAIESHYGWLATRLYRAFRPKCLKVDVGWRWRRMCCIPIVLIFELALVTVVTGISLTVAYFTFADEKEKEHILVALYVIAAVMGTLICTHLHVLAKVFVSLFTSHIRVLKRAVRSSESAPLTMLGAEVAVMTDMVKCLDAFTNQQSRLVGVIDALDSCDTERILTLLNAVQTLLSSPNRPFVLLISVDPHVIAKAAEANSRRLFTEGGIGGHDFLRNLVHLPVYLQNSGLRKVQRAQMTALLFKRSGGGDYQTDDGPTLGHSVSARRLSNASEIISSQEKLRGPARGGGGKKLRLSESVASSTGSNLHRLGQNPQTVLDLSRIVLTDDYFSDVNPRSMRRLMNVIYITVRLLKAFQIEFSWYRLSSWINLTEQWPLRASMIVLHHDQFMDSNADESVSLQSVYEKLRPKLAYLREAAPLLELDRDERKLDAFLQLHKSDLLVADLRIFLPFTINLDPYLRKVLKEDQQTIEDEGSLVIQARPSVSNTMRQFPAPTTYVPSPQAYPPYQMFQNEYPANELRSRNLSTSTEPVTPLINSPSDSFGDDILQTKLTDLTVEGVISLLDRIEDMKPALPKLAPVLRENAINGRVLKHCDMPDLKSVLGLSFGHWELFRLLITTLRECERLPRKQQRQQQQPGALEAPSNVPMIKDVTDALMQPPRESLSRKNSVSHMEKQVTLEEQMICGTLQTLNEEAYEDVASSERPSPTGEMLAAVAQLQLAPIRESSEFGSPSDDQKQYGVKISNNNNNNQYLHAEYNRSVSSHSLQSLSTLVGAPVGHGGSGGSHLHLGNGNDLSDSTLDLMHVDSVFGGGGGGGYHRASRQISISSELLHESSSLPMVVVIPNASGEQAYDDTKL

(19) United States
(12) Patent Application Publication
(10) Pub. No.: US 2003/0166056 A1
(43) Pub. Date: Sep. 4, 2003

(54) Transmembrane protein as a downstream target of neurotrophin and ephrin receptor tyrosine kinases, DNA encoding same and monoclonal antibodies thereto

(75) Inventors: Moses V. Chao, New York, NY (US); Haeyoung Kong, La Jolla, CA (US)

Correspondence Address: BROWDY AND NEIMARK, P.L.L.C., 624 NINTH STREET, NW, SUITE 300, WASHINGTON, DC, 20001-5303, US

(73) Assignee: New York University, New York, NY (US)

(21) Appl. No.: 10/021,571

(22) Filed: Dec. 19, 2001

Related U.S. Application Data
(60) Provisional application No. 60/256,909, filed on Dec. 21, 2000.

(51) Int. Cl. C12Q 1/68; C07H 21/04; C12P 21/02; C12N 5/06; C07K 14/435

(52) U.S. Cl. 435/69.1; 435/320.1; 435/325; 530/350; 536/23.2; 435/6

(57) ABSTRACT

The present invention relates to an ankyrin repeat-rich membrane spanning (ARMS) polypeptide which is a downstream target of neurotrophin and ephrin receptor tyrosine kinases. This ARMS polypeptide contains 1715 amino acid residues including four transmembrane domains, multiple ankyrin repeats, a sterile α motif domain, and a PDZ-binding motif. Immunofluorescence studies with anti-ARMS antibodies have found ARMS to be localized discreetly to the growth cone of neurons.

### CROSS-REFERENCE TO RELATED APPLICATIONS

The present application claims priority under 35 U.S.C. §119(e) from U.S. provisional application No. 60/256,909, filed Dec. 21, 2000, the content of which is hereby incorporated entirely by reference.

### GOVERNMENT LICENSE RIGHTS

The experiments performed in this application were supported in part by the National Institutes of Health, Grant Nos. NS-21072, HD-23315, and NS-10489. The U.S. Government has a paid-up license in this invention and the right in limited circumstances to require the patent owner to license others on reasonable terms as provided for by the terms of Grant Nos. NS-21072, HD-23315, and NS-10489 awarded by the National Institutes of Health.

### BACKGROUND OF THE INVENTION

1. Field of the Invention

The present invention relates to a transmembrane protein which is a downstream target of neurotrophin and ephrin receptors for phosphorylation.

2. Description of the Related Art

The formation of the nervous system requires appropriate connectivity of neurons and their targets both spatially and temporally. Two families of proteins that mediate these actions are the neurotrophins and ephrins. Neurotrophins play a prominent role in the development of the vertebrate nervous system by influencing cell survival, differentiation and cell death events (Levi-Montalcini, 1987; Lewin and Barde, 1996). Neurotrophins also exhibit acute regulatory effects upon neurotransmitter release, synaptic strength and connectivity (Thoenen, 1995; Bonhoeffer, 1996; McAllister et al., 1999). In addition to promoting axonal (Patel et al., 2000) and dendritic branching, neurotrophins serve as chemoattractants for extending growth cones in vitro (Letourneau, 1978; Gundersen and Barrett, 1979; Gallo et al., 1997).

Ephrins comprise another class of ligands that function in axon guidance, cell migration, axon fasciculation, boundary formation, topographic mapping, and morphogenesis (Frisen et al., 1999). Ephrins are a family of eight proteins that are found associated with the plasma membrane, either through a GPI linkage (as seen in the A subfamily) or as transmembrane proteins (as seen in the B subfamily). Ephrins signal via receptor protein tyrosine kinases, but the biological outcomes are distinct from mitogenic factors such as platelet-derived growth factor (PDGF) and epidermal growth factor (EGF), both of which transmit signals through tyrosine phosphorylation (Bruckner and Klein, 1998; Flanagan and Vanderhaeghen, 1998; Holland et al., 1998). Ephrin receptor-associated molecules such as Crk, Nck, RasGAP, and Fyn are proposed links between the receptor and downstream events such as cell adhesion and cytoskeletal changes. In addition, Grb2, Grb10, and the p85 subunit of phosphatidylinositol-3-kinase (PI-3 kinase) are utilized in ephrin receptor signal transduction (Mellitzer et al., 2000).

The neurotrophins, NGF, BDNF, NT-3 and NT-4/5, exert their effects through two classes of receptors (Kaplan and Miller, 2000). TrkA, TrkB, and TrkC serve as receptors for NGF, BDNF and NT-4, and NT-3, respectively (Chao, 1992a). The p75 receptor is a member of the TNF receptor superfamily (Smith et al., 1994) and binds to all neurotrophins. Most central and peripheral neurons express p75 together with one or more of the Trks. The p75 receptor, when co-expressed with TrkA, provides a positive influence upon Trk function (Barker and Shooter, 1994; Verdi et al., 1994) and determines specificity of neurotrophin binding and responsiveness (Benedetti et al., 1994; Bibel et al., 1999; Brennan et al., 1999). An association of p75 and Trk receptors has been detected by co-precipitation (Huber and Chao, 1995; Gargano et al., 1997; Bibel et al., 1999) and co-patching of these receptors has been observed using fluorescent labeled antibodies (Ross et al., 1996).

Receptor tyrosine kinases frequently utilize a number of common intracellular signaling components such as phospholipase C-γ, PI-3 kinase and adaptor proteins such as Shc and Grb2. Common to many of these proteins is their ability to bind to phosphorylated tyrosines via domains such as the SH2 and phosphotyrosine-binding (PTB) domains. How these shared signaling components lead to different biological outcomes is not well understood (Chao, 1992a). Possible mechanisms include receptor utilization of substrates in a differential manner (e.g., differential association/dissociation kinetics), competition for binding between different substrates (Meakin et al., 1999), or recruitment of unique target proteins, such as rAPS and SH2-B (Qian et al., 1998).

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

The present invention provides a polypeptide which is a transmembrane protein that is highly expressed in vertebrate central and peripheral nervous systems. This polypeptide, which associates with TrkA and p75 neurotrophin receptors, is a target for phosphorylation by neurotrophin and ephrin receptor tyrosine kinases, enhances neurotransmitter release, and modulates the clustering of proteins involved in ion channel formation, includes the amino acid sequence of: (A) SEQ ID NO:2; (B) SEQ ID NO:4; (C) a fragment of the polypeptide of SEQ ID NO:2; (D) a fragment of the polypeptide of SEQ ID NO:4; (E) a variant polypeptide which is at least 95% identical to SEQ ID NO:2; (F) a variant polypeptide which is at least 95% to SEQ ID NO:4; or (G) a functional derivative or a salt of (A), (B), (C), (D), (E), or (F).

The present invention also provides an isolated nucleic acid which encodes the polypeptide of the present invention or which hybridizes under stringent conditions to the complement of the nucleotide sequence encoding the polypeptide of the present invention a vector, a transformed host cell containing such a nucleic acid, and a method for producing the polypeptide of the present invention.

Further provided by the present invention is a molecule containing the antigen-binding portion of an antibody specific for the polypeptide of the present invention.

A still further aspect of the present invention provides a method for visualizing the growth cone of neurons, where the polypeptide of the present invention serves as a marker for growth cones.

### BRIEF DESCRIPTION OF THE DRAWINGS

FIG. 1 shows the predicted topology of ARMS. Transmembrane domains and various intracellular motifs are depicted.

FIG. 2 shows the amino acid sequence and comparison of rat (SEQ ID NO:2) and human (SEQ ID NO:4) ARMS proteins. Dash-lined residues denote 11 contiguous ankyrin repeats; bold-faced tyrosine (Y) residues (at positions 399, 409, 441, 444, and 466 of the rat sequence) are evolutionarily conserved among human, rat, Drosophila, and C. elegans; boxed residues are the putative transmembrane domains; italicized residues denote the polyproline stretch; shadowed residues constitute the SAM domain (aa1152-1221); carboxy-most three asterisked amino acids (SIL) encode a PDZ-binding motif.

FIG. 3 shows the comparison of various cytoplasmic regions of rat (r), human (h), Drosophila (d) and C. elegans (w) ARMS. 1, amino terminal region between the ankyrin repeats and the first transmembrane domain with bold-faced, evolutionarily conserved tyrosines (Y); 2, cytoplasmic region between transmembrane domains 2 and 3; 3 and 4, two carboxy terminal regions; 5, the SAM domain. Sequences for wARMS and dARMS were obtained from accession numbers Z68760 and AAF46710, respectively. The sequence for hARMS was determined from overlapping ESTs with accession numbers BAA86564 and CAB63746. (symbols: “*” identity, “:” strongly similar, “.” weakly similar). The residue numbering of the various cytoplasmic regions corresponds to the residue numbering of the amino acid sequences of rARMS (SEQ ID NO:2), hARMS (SEQ ID NO:4), dARMS (SEQ ID NO:8), and wARMS (SEQ ID NO:6).

FIGS. 4A and 4B show a Northern analysis of ARMS (FIG. 4A) and a methylene blue staining of the 28S ribosomal band as a loading control (FIG. 4B). A single transcript of 7.0 kb was detected by Northern analysis using a ³²P-labeled ARMS cDNA probe (FIG. 4A). Each lane contained 20 μg of total RNA (with the exception of pancreas and DRG lanes which contained <10 μg each) extracted from various rat tissue.

FIGS. 5A-5D show the distribution of ARMS mRNA in the adult rat central nervous system by in situ hybridization. A ³³P-labeled cRNA probe was used to assess ARMS mRNA expression. Areas of intense labeling include the mitral cell layer of the olfactory bulb (OB; FIG. 5A), all regions of the hippocampus (HP; FIG. 5B), the Purkinje cell layer of the cerebellum (CB; FIG. 5C), grey matter—most notably in the ventral horn—of the spinal cord (SC; FIG. 5D).

FIGS. 6A and 6B show the expression in adult rat dorsal root ganglion (DRG) by in situ hybridization. A ³³P-labeled cRNA probe was used to assess mRNA distribution in DRG as depicted in the dark field image (left). The majority of cell bodies of the DRG were positive for ARMS mRNA expression, but notable absences of expression were localized to the large diameter DRG cell bodies as depicted by the arrows in the dark field and the corresponding phase (FIG. 6B) photographs. (scale bars=1 mm)

FIGS. 7A-7C show expression of ARMS mRNA by in situ hybridization of ARMS in embryonic day 14 (E14) rat. In a coronal section through the midsection of an E14 rat, only spinal cord (sc) and dorsal root ganglion (drg) were positive for ARMS (FIG. 7A). In situ hybridization of ARMS in a midsagittal section (FIG. 7B) and a more lateral section (FIG. 7C) of E14 rat. ARMS mRNA expression was restricted to various brain regions such as the cortex (cx), hippocampus (hp), pons, medulla (med), basal telencephalon (bt), principal and spinal trigeminal nucleus (tn), superior and inferior colliculus (clc) and spinal cord (sc). Multiple ganglia expressed ARMS mRNA, such as the dorsal root ganglion (drg), trigeminal ganglion (tg), geniculate ganglion (gg), vestibular ganglion (vg), and superior cervical ganglion (scg). (white scale bars=1 mm; black scale bar=50 μm)

FIGS. 8A-8B show the interaction of p75 with ARMS. HEK293T cells were co-transfected with cDNAs encoding full length ARMS, HA-tagged p75, ARMS plus p75, or empty vector. Cells lysates were immunoprecipitated with anti-ARMS 892 antiserum and immunoblotted with anti-HA (FIG. 8A). Expression of p75 receptors was confirmed by immunoblotting with anti-p75 (9992; FIG. 8B).

FIGS. 9A-9D show coprecipitation of TrkA and ARMS (FIG. 9A) and colocalization of TrkA and ARMS (FIGS. 9B-9D). In FIG. 9A, PC12 615 cells were treated for 10 minutes and 25 hours with NGF (100 ng/ml). Lysates were prepared and subjected to immunoprecipitation with anti-Trk C-14 antibody, followed by immunoblot with anti-ARMS antibody. Normal rabbit IgG was used as a negative control. In FIGS. 9B-9D, immunofluorescence analysis of ARMS and TrkA receptor in sympathetic neurons is shown. SCG sympathetic neurons were grown in the presence of 150 ng/ml NGF, fixed and immunostained as described in the Methods section of Example 1. The ARMS protein and the TrkA receptor were subjected to double immunostaining using an anti-ARMS antiserum (FIG. 9B) and an anti-Trk B-3 (FIG. 9C) monoclonal antibody and analyzed by confocal microscopy. The signal observed in FIG. 9D demonstrates overlap of the two signals (overlay) from immunostaining with anti-ARMS anti-serum and with an anti-TrkB monoclonal antibody. The arrow indicates cell surface co-localization of ARMS and TrkA.

FIGS. 10A and 10B show tyrosine phosphorylation of ARMS. In FIG. 10A, phosphorylation of ARMS by NGF in PC12 cells is rapid and can be blocked by K252a. The antiserum 892 was used to immunoprecipitate endogenously expressed ARMS from PC12 615 cell lysates. Anti-phosphotyrosine antibody, pY99 was used to assess tyrosine phosphorylation of the immunoprecipitated ARMS. Within 1 minute of NGF treatment, phosphorylation of ARMS could be detected, suggesting a direct phosphorylation by TrkA. Furthermore, 100 nM K252a potently blocked ARMS phosphorylation (top). In lysates of the same samples, TrkA autophosphorylation is shown using pY99 (bottom). The time course of ARMS phosphorylation by NGF in PC12 cells is shown in FIG. 10B. The phosphorylation peaks within 10 minutes (m) and is sustained for at least 25 hours (h) (top). Reprobing of the same blot with 892 demonstrated equivalent levels of immunoprecipitated ARMS from the various lysates (bottom).

FIG. 11 shows specificity of ARMS phosphorylation. Phosphorylation of ARMS is specifically induced upon NGF, but not EGF, treatment of PC12 615 cells. Two time points, 10 minutes and 2 hours, were examined for tyrosine phosphorylation of ARMS using the following conditions: no ligand (CTRL), 50 ng/ml EGF and 100 ng/ml NGF. To demonstrate the specificity of the ARMS antiserum, 892 (I), preimmune antiserum (P) was used in parallel immunoprecipitations (IP). The lower panel shows the amount of ARMS protein that was immunoprecipitated from the various lysates.

FIG. 12 shows the effects of other neurotrophins. The neurotrophins BDNF and NT-4/5 induce phosphorylation of ARMS through the TrkB receptor. PC12 cells stably expressing TrkB were treated with either 100 ng/ml BDNF or 100 ng/ml NT4/5 and the phosphorylation of ARMS was measured as described in FIG. 10A. BDNF, and to a lesser extent, NT4/5, were able to induce tyrosine phosphorylation of ARMS. The bottom panel depicts immunoprecipitated ARMS from each lysate.

FIG. 13 shows induction of ARMS phosphorylation in hippocampal neurons by BDNF. Primary cultures of E17 hippocampal neurons were prepared and treated with 50 ng/ml BDNF for the indicated times. Phosphorylation of ARMS was assessed by immunoprecipitation with anti-ARMS 892 antiserum and Western blotting with anti-phosphotyrosine pY99 antibody (top panel). Equal amounts of ARMS protein were immunoprecipitated from each lysate as shown with reprobing the same blot with 892.

FIGS. 14A and 14B show the effects of ephrins. In FIG. 14A, ephrin B2 induces ARMS tyrosine phosphorylation in NG108-15 cells expressing EphB2 receptor. Lysates were made from untreated or ligand-stimulated NG108-15 cells (using aggregated ephrin B2, 30-40 minutes) and immunoprecipitated with 892 antiserum. Tyrosine phosphorylation was assessed with pY99 in subsequent Western blots. Equivalent amounts of ARMS were immunoprecipitated as shown in the lower panel. In FIG. 14B, tyrosine phosphorylation of ARMS by ephrin B2 peaks at 30 minutes. Thus, the time course of ARMS tyrosine phosphorylation closely parallels that of receptor autophosphorylation.

FIGS. 15A and 15B show ARMS/Trk receptor interaction. Expression plasmids containing full length cDNAs for ARMS and TrkA, TrkB and TrkC receptors were transiently transfected into HEK293 cells (2×10⁶ cells/plate) following the calcium-phosphate method. ARMS, Trk and EGF receptor expression were detected by immunoblotting. Cells were lysed in 1% NP-40 lysis buffer containing 20 mM Tris-HCl pH 8, 150 mM NaCl, 1% NP-40, 2 mM EDTA and protease inhibitors (0.15 units/ml aprotinin, 20 uM leupeptin and 1 mM phenylmethylsulphonylfluoride), at 4° C., for 30 min. Immunoprecipitation was performed for 3 hours at 4° C. using 2-3 mg of total protein extract and the Flag agarose-conjugated antibody (Sigma, St. Louis, Mo.). After several washes, immunoprecipitates were analyzed by SDS-PAGE followed by Western blot with different antibodies e.g., antibodies against TrkA, TrkB, TrkC, Arms, or EGFR. Reactive protein bands were visualized by enhanced chemiluminescence detection (Amersham Corp., Piscataway, N.J.).

FIGS. 16A and 16B show a PC12 Immunofluorescence analysis. PC12 cells with (FIG. 16A) or without (FIG. 16B) NGF treatment cultured in Lab-Tek chamber slides (Nalge Nunc International) coated with collagen and poly-L-lysine were fixed with paraformaldehyde and permeablized with cold methanol. Cells were blocked with PBS containing 10% FCS and incubated with purified antibody (3 μg/ml) against the C-terminus of ARMS protein, 892 (Example 1). Primary antibodies were visualized using fluorescence-conjugated secondary antibodies (FITC-conjugated goat anti-rabbit IgG; Jackson Laboratories). Images were collected on a Leica confocal microscope (Nussloch, Germany) and show that ARMS is localized at neurite tips in PC12 cells after NGF treatment.

FIGS. 17A-17F show immunolocalization of ARMS and VAMP-2, a synaptic vesicle marker, in hippocampal neurons. Primary cultures of hippocampal neurons were obtained from rats E17-19 and maintained with Neurobasal medium (Gibco) supplemented with B-27 and 0.4 mM glutamine in Lab-Tek chamber slides (Nalge Nunc International) coated with poly-L-lysine. The cells were fixed with paraformaldehyde, permeablized with cold methanol and then blocked with PBS containing 10% FBS, 10% normal goat serum and 5% BSA for at least 30 minutes and incubated with anti-ARMS antibody 892 (FIG. 17A) and VAMP-2 (FIG. 17B) antibodies in blocking solution at room temperature or 4° C. Primary antibodies were detected using fluorescence-conjugated secondary antibodies (FITC-conjugated goat anti-rabbit IgG and rhodamine-conjugated goat anti-mouse IgG (Jackson Laboratories)). Images were collected on a Leica confocal microscope. FIG. 17C is a merged image of FIGS. 17A and 17B). FIGS. 17D-17F represent an enlargement of the staining in the top FIGS. 17A-17C, respectively, with the white arrows designating tips of processes

FIG. 18 shows the localization of ARMS in axons and growth cones of hippocampal neurons. Cultures of hippocampal neurons were assessed for ARMS expression by indirect immunofluorescence, as described above for FIG. 17A. A concentration of ARMS protein was found at the growth cone (white arrow) and along the axon in a punctate distribution.

FIG. 19 shows the interaction between ARMS and PDZ-containing proteins GRIP1, GRIP2 and PICK1. Expression plasmids containing myc-epitope tagged GRIP1, GRIP2 or PICK1 were cotransfected with a full length cDNA for ARMS in HEK293 cells. Cells were lysed in 1% NP-40 lysis buffer and immunoprecipitation was carried out with anti-myc antibodies, followed by Western blot for the ARMS protein.

FIG. 20 shows a proposed schematic model of interactions between Trk receptors, ARMS, PDZ-containing proteins and glutamate receptors.

### DETAILED DESCRIPTION OF THE INVENTION

The present inventors have discovered a novel transmembrane protein, designated ARMS (for Ankyrin Repeat-Rich, Membrane Spanning), in rats and humans which is a downstream target/substrate of neurotrophin and ephrin receptor tyrosine kinases, Trk and Eph, respectively. This ARMS protein/polypeptide according to the present invention was found to have 1,715 amino acids (SEQ ID NO:2 for rat ARMS and SEQ ID NO:4 for human ARMS) containing four putative transmembrane domains, multiple ankyrin repeats, a SAM domain, and a potential PDZ-binding motif.

The ARMS protein of the present invention is a substrate for tyrosine phosphorylation by the Trk neurotrophin family of tyrosine kinase receptors. These receptors mediate the actions of neurotrophins (NGF, BDNF, NT-3 and NT-4), which are required for the survival and differentiation of neurons during development and after injury. Moreover, neurotrophin receptor signaling is required for a form of memory known as long term potentiation (LTP), formation of synapses, axonal targeting and hyperalgesia (pain). The ARMS protein is specifically a target for neurotrophins and ephrins, which are also responsible for axon guidance. Unlike other phosphotyrosine proteins, the ARMS protein is a specific target for neurotrophins and ephrins, and not for other polypeptide growth factors which use tyrosine kinase receptors.

Because of its specificity and highly neuronal expression pattern, ARMS is a useful indicator of the biological activity of neurotrophins and ephrins. The ARMS gene is expressed in postmitotic neurons during the stage of development in which extensive axon pathfinding is occurring and is also expressed during adulthood in highly “plastic” regions of the brain such as the hippocampus, cortex, Purkinje cells of the cerebellum, the olfactory bulb, and of the spinal cord motor neurons, regions which are enriched in Trk and Eph receptors.

The most significant commonality of these neuronal populations is their ability to undergo continued synaptic changes throughout adult life. Neurons of the olfactory bulb are continually renewed and hence must form new synapses; hippocampal neurons can undergo synaptic remodeling and LTP; Purkinje cell dendrites are highly plastic due to their constant structural remodeling; and motor neurons have the capacity to regenerate and to form new synapses with peripheral targets in adults. Peripheral neurons have regenerative properties throughout adulthood, and this process requires axon outgrowth and new synapse formation. Neurotrophins and ephrins are likely candidates in spinal cord regeneration (Frisen et al., 1992; Miranda et al., 1999), and the present inventors believe that ARMS is also utilized in this process.

The ARMS gene is also expressed in sensory, geniculate, vestibular and sympathetic neurons in the peripheral nervous system. These neural regions control hearing, vision, taste, smell, cardiovascular and motor functions. The ARMS protein is a very abundant neuronal-specific protein that contains consensus docking sites for ion channels and other signal transduction proteins. From immunofluorescence and confocal microscopic studies, there is a dramatic change in the distribution of ARMS in both hippocampal neurons and PC12 cells after treatment with neurotrophins BDNF or NGF, demonstrating that the ARMS protein is localized discretely at growth cones and in synaptic regions of neuronal membranes where neuronal activities are located.

The predicted integral membrane structure of ARMS led the present inventors to be believe that ARMS may function as an ion channel. The four predicted transmembrane domains of the ARMS protein and its overall structure is reminiscent of the TRP family of ion channels and the capsaicin receptor, VR1 (Caterina et al., 1997; Harteneck et al., 2000). These channels contain six transmembrane domains with intracellular amino and carboxy termini and amino terminal ankyrin repeats. Although ARMS channel activity was not detected in gene transfer experiments, it remains possible that this protein may also serve as a subunit of a channel, or more likely, is involved in clustering or maintenance of ion channels. Such a possibility has been suggested by the ability of the neurotrophin BDNF to elicit hippocampal, cortical, and cerebellar depolarization on a very rapid time scale (Kafitz et al., 1999), presumably through activation of sodium channels. While the mechanism of this activation is unknown, phosphorylation of ARMS by TrkB suggests a role for ARMS in neurotrophin-mediated regulation of neuronal activity, such as changes in electrophysiological activity of neurons.

Furthermore, the presence of several protein interaction domains strongly supports a role for ARMS in recruiting proteins to Trk receptor tyrosine kinases. Indeed, co-immunoprecipitation studies indicate that ARMS may be an integral part of a higher order Trk-p75 receptor complex. These interactions may not be limited to neurotrophin signaling, as the Eph receptor family is also capable of phosphorylating the ARMS protein.

Several neuronal proteins were found to bind to the ARMS protein. These proteins include synembryn, synaptotagmin, a-catenin and cdcRel, which are all involved in synaptic vesicle trafficking. ARMS appears to be involved in the neurotransmitter vesicle movements in the nerve cells and may be involved in directing the release of neurotransmitters from neurons. These neurotransmitters may be acetylcholine or dopamine. Another protein that was discovered to interact with the ARMS protein is the regulatory subunit of K⁺channels. This indicates that ARMS may be associated with ion channel activities, which give rise to changes in neuronal activity (action potentials). The significance of these findings stem from the potent effects of neurotrophins upon synaptic plasticity. The phosphorylation of ARMS by Trk and Eph receptors provides a mechanism for transmitting information from neurotrophins to specific regions of synaptic activity in nerve cells.

One aspect of the present invention is therefore directed to an ARMS polypeptide which associates with TrkA and p75 neurotrophin receptors and is a target for phosphorylation by neurotrophin and ephrin receptor tyrosine kinases. This polypeptide of the present invention contains the rat ARMS amino acid sequence of SEQ ID NO:2, a fragment of the polypeptide of SEQ ID NO:2 or a variant thereof which is at least 95% identical to SEQ ID NO:2, the human ARMS amino acid sequence of SEQ ID NO:4, a fragment of the polypeptide of SEQ ID NO:4 or a variant thereof which is at least 95% identical to SEQ ID NO:4, or a derivative or salt of any of the above.

Fragments of the full-length ARMS polypeptide of SEQ ID NO:2 or SEQ ID NO:4 are intended to cover any active fragment that retains the biological activity of the full-length ARMS polypeptide. For example, fragments can be readily generated from the full-length ARMS polypeptide where successive residues can be removed from either or both the N-terminus or C-terminus of ARMS, or from peptides obtained thereof by enzymatic or chemical cleavage of the polypeptide. Thus, multiple substitutions are not involved in screening for active fragments of ARMS. If the removal of one or two amino acids from one end or the other does not affect the biological activity after testing in assays described in the Examples and Figures herein, such truncated polypeptides are considered to be within the scope of the present invention. Further truncations can then be carried out until it is found where the removal of another residue destroys the biological activity.

Preferably, such active fragments of ARMS would contain the PDZ-binding motif in the cytoplasmic C-terminal portion of ARMS and further either contain one or more transmembrane domains of ARMS or be fused as a fusion polypeptide to a transmembrane domain, other than a transmembrane domain of ARMS, which allows a fragment of ARMS to retain the biological activity of the full-length ARMS polypeptide.

The term “variant” is intended to encompass a variant of the native sequence of SEQ ID NO:2 or SEQ ID NO:4, or of a biologically active fragment thereof, which has an amino acid sequence having at least 85% identity, preferably at least 90% identity, or more preferably at least 95% identity to the native sequence and retains the biological activity thereof. As would be appreciated by those of skill in the art, this term covers naturally-occurring variants as a preferred embodiment.

“Functional derivatives” as used herein covers chemical derivatives which may be prepared from the functional groups which occur as side chains on the residues or the N- or C-terminal groups, by means known in the art, and are included in the invention as long as they do not destroy the biological activity of the corresponding polypeptide/protein as described herein. Derivatives may have chemical moieties, such as carbohydrate or phosphate residues, provided such derivative have the same biological activity.

Suitable derivatives may include aliphatic esters of the carboxyl groups, amides of the carboxyl groups by reaction with ammonia or with primary or secondary amines, N-acyl derivatives or free amino groups of the amino acid residues formed with acyl moieties (e.g., alkanoyl or carbocyclic aroyl groups), O-acyl derivatives of free hydroxyl group (e.g., that of seryl or threonyl residues) formed with acyl moieties, or phosphorylated derivatives of free hychoxyl groups in serine and threonine residues. Such derivatives may also include for example, polyethylene glycol side-chains which may mask antigenic sites and extend the residence of the complex or the portions thereof in body fluids.

Non-limiting examples of such derivatives are described below.

Cysteinyl residues most commonly are reacted with alpha-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, alpha-bromo-beta-(5-imidazoyl)propionic acid, chloroacetyl phosphate, -alkylmaleimides, 3-nitro-2-pyridyl disulfide, methyl-2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Parabromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2, 4-pentanedione; and transaminase-catalyzed reaction with glyoxylate.

Arginyl residues are modified by reaction with one or several conventional reagents, among them phenylglyoxal, 2,3-butanedione, 1,2-cyclodexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues per se has been studied extensively, with particular interest in introducing spectral labels into tyrosyl residues by reaction with aromatic diazonium compounds or tetranitromethane. Most commonly, N-acetylimidazole and tetranitromethane are used to form O-acetyl tyrosyl species and 3-nitro derivatives, respectively.

Carboxyl side groups (aspartyl or glutamyl) are selectively modified by reaction with carbodiimides (R′—N—C—N—R′) such as 1-cyclohexyl-3-[2-morpholinyl-(4-ethyl)]carbodiimide or 1-ethyl-3-(4-azonia-4,4-dimethlypentyl) carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues. Alternatively, these residues are deamidated under mildly acidic conditions. Either form of these residues falls within the scope of this invention.

The term “derivatives” is intended to include only those derivatives that do not change one amino acid to another of the twenty commonly-occurring natural amino acids.

The term “salts” herein refers to both salts of carboxyl groups and to acid addition salts of amino groups of the complex of the invention or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must have substantially similar biological activity to the polypeptide of the invention.

Another aspect of the present invention is directed to a molecule which contains the antigen-binding portion of an antibody specific for the ARMS polypeptide of the present invention. It should be understood that the term “antibody” is intended to include intact antibodies, such as polyclonal antibodies or monoclonal antibodies (mAbs), as well as proteolytic fragments thereof such as the Fab or F(ab′)₂ fragments. Furthermore, the DNA encoding the variable region of the antibody can be inserted into DNA encoding other antibodies to produce chimeric antibodies (see, for example, U.S. Pat. No. 4,816,567).

Single chain antibodies can also be produced and used. Single chain antibodies can be single chain composite polypeptides having antigen binding capabilities and comprising a pair of amino acid sequences homologous or analogous to the variable regions of an immunoglobulin light and heavy chain (linked V_{H}-V_{L} or single chain F_{V}). Both V_{H} and V_{L} may copy natural monoclonal antibody sequences or one or both of the chains may comprise a CDR-FR construct of the type described in U.S. Pat. No. 5,091,513 (the entire contents of which are hereby incorporated herein by reference). The separate polypeptides analogous to the variable regions of the light and heavy chains are held together by a polypeptide linker. Methods of production of such single chain antibodies, particularly where the DNA encoding the polypeptide structures of the V_{H} and V_{L} chains are known, may be accomplished in accordance with the methods described, for example, in U.S. Pat. Nos. 4,946,778, 5,091,513 and 5,096,815, the entire contents of each of which are hereby incorporated herein by reference.

A “molecule which contains the antigen-binding portion of an antibody” is intended to include not only intact immunoglobulin molecules of any isotype and generated by any animal cell line or microorganism, or generated in vitro, such as by phage display technology for constructing recombinant antibodies, but also the antigen-binding reactive fraction thereof, including, but not limited to, the Fab fragment, the Fab′ fragment, the F(ab′)₂ fragment, the variable portion of the heavy and/or light chains thereof, and chimeric or single-chain antibodies incorporating such reactive fraction, as well as any other type of molecule or cell in which such antibody reactive fraction has been physically inserted, or molecules developed to deliver therapeutic moieties by means of a portion of the molecule containing such a reactive fraction. Such molecules may be provided by any known technique, including, but not limited to, enzymatic cleavage, peptide synthesis or recombinant techniques.

The polypeptide of the present invention can be used as a marker for neuronal cells which have the ability to undergo continued synaptic changes through adult life or for the presence and distribution (i.e, clustering and aggregation) of the ARMS polypeptide in such neuronal cells. More specifically, as demonstrated in Example 2, the polypeptide of the present invention is a marker for growth cones as ARMS was found to be localized discretely at growth cones and in the synaptic regions of neurons. It was also found that ARMS co-localized with Vamp-2, a synaptic vesicle marker, and can therefore be used for the same purposes as Vamp-2.

A molecule which contains the antigen-binding portion of an antibody specific for the ARMS polypeptide of the present invention can be labeled with a labeling compound and imaged in vitro or in vivo as is well-known in the diagnostic and imaging art. As a preferred embodiment, this molecule can be used in a method for visualizing the growth cone of neurons which method involves contacting a molecule containing the antigen-binding portion of an antibody specific for the ARMS polypeptide of the present invention with neurons to detect the presence of the polypeptide of SEQ ID NO:2 or SEQ ID NO:4 as a marker for the growth cone of neurons. The growth cone of neurons can then be visualized when the molecule containing the antigen-binding portion of an anti-ARMS antibody is detectably labeled, either directly or indirectly, i.e., through the binding of a secondary detectably labeled antibody to the anti-ARMS antibody.

Further aspects of the present invention is directed to a nucleic acid or polynucleotide which encodes the ARMS polypeptide of the present invention, a vector containing the nucleic acid, and a host cell transformed with such a nucleic acid. Preferably, the nucleic acid includes the nucleotide sequence encoding either the rat ARMS polypeptide of SEQ ID NO:2 or the human ARMS polypeptide of SEQ ID NO:4 or fragments thereof. More preferably, the nucleic acid includes the nucleotide sequence of SEQ ID NO:l or SEQ ID NO:3.

The nucleic acid according to the present invention is intended to encompass nucleic acids which specifically hybridizes under stringent conditions to the complement of either SEQ ID NO:1 or SEQ ID NO:3. Stringency conditions are a function of the temperature used in the hybridization experiment and washes, the molarity of the monovalent cations in the hybridization solution and in the wash solution(s) and the percentage of formamide in the hybridization solution. In general, sensitivity by hybridization with a probe is affected by the amount and specific activity of the probe, the amount of the target nucleic acid, the detectability of the label, the rate of hybridization, and the duration of the hybridization. The hybridization rate is maximized at a Ti (incubation temperature) of 20-25° C. below Tm for DNA:DNA hybrids and 10-15° C. below Tm for DNA:RNA hybrids. It is also maximized by an ionic strength of about 1.5M Na⁺. The rate is directly proportional to duplex length and inversely proportional to the degree of mismatching.

Specificity in hybridization, however, is a function of the difference in stability between the desired hybrid and “background” hybrids. Hybrid stability is a function of duplex length, base composition, ionic strength, mismatching, and destabilizing agents (if any).

The Tm of a perfect hybrid may be estimated for DNA:DNA hybrids using the equation of Meinkoth et al (1984), as

Tm=81.5° C.+16.6(log M)+0.41 (%GC)−0.61(% form)−500/L

and for DNA:RNA hybrids, as

Tm=79.8° C.+18.5 (log M)+0.58 (%GC)−11.8(%GC)²−0.56(% form)−820/L

where

M, molarity of monovalent cations, 0.01-0.4 M NaCl,

%GC, percentage of G and C nucleotides in DNA, 30%-75%,

% form, percentage formamide in hybridization solution, and

L, length hybrid in base pairs.

Tm is reduced by 0.5-1.5° C. (an average of 1° C. can be used for ease of calculation) for each 1% mismatching.

The Tm may also be determined experimentally. As increasing length of the hybrid (L) in the above equations increases the Tm and enhances stability, the full-length rat gene sequence can be used as the probe.

Filter hybridization is typically carried out at 68° C., and at high ionic strength (e.g., 5-6×SSC), which is non-stringent, and followed by one or more washes of increasing stringency, the last one being of the ultimately desired high stringency. The equations for Tm can be used to estimate the appropriate Ti for the final wash, or the Tm of the perfect duplex can be determined experimentally and Ti then adjusted accordingly.

Hybridization conditions should be chosen so as to permit allelic variations, but avoid hybridizing to other genes. In general, stringent conditions are considered to be a Ti of 5° C. below the Tm of a perfect duplex, and a 1% divergence corresponds to a 0.5-1.5° C. reduction in Tm. Typically, rat clones were 95-100% identical to database rat sequences, and the observed sequence divergence may be artifactual (sequencing error) or real (allelic variation). Hence, use of a Ti of 5-15° C. below, more preferably 5-10° C. below, the Tm of the double stranded form of the probe is recommended for probing a rat cDNA library with a rat DNA probe or a human cDNA library with a human DNA probe.

As used herein, highly stringent conditions are those which are tolerant of up to about 5% sequence divergence. Without limitation, examples of highly stringent (10° C. below the calculated Tm of the hybrid) conditions use a wash solution of 0.1×SSC (standard saline citrate) and 0.5% SDS at the appropriate Ti below the calculated Tm of the hybrid. The ultimate stringency of the conditions is primarily due to the washing conditions, particularly if the hybridization conditions used are those which allow less stable hybrids to form along with stable hybrids. The wash conditions at higher stringency then remove the less stable hybrids. A common hybridization condition that can be used with the highly stringent wash conditions described above is hybridization in a solution of 6×SSC (or 6×SSPE), 5×Denhardt's reagent, 0.5% SDS, 100 μg/ml denatured, fragmented salmon sperm DNA at an appropriate incubation temperature Ti.

A still further aspect of the present invention is directed to a method for producing a polypeptide of the present invention by culturing in a nutrient medium a host cell transformed with the nucleic acid according to the present invention, as discussed above, to produce and express the polypeptide and then recovering the produced polypeptide.

Having now generally described the invention, the same will be more readily understood through reference to the following examples which are provided by way of illustration and is not intended to be limiting of the present invention.

### EXAMPLE 1

To define proteins associated with the neurotrophin receptors, the intracellular domain of the p75 neurotrophin receptor was used as bait in a two-hybrid screen of a dorsal root ganglion library. The properties of the novel transmembrane protein according to the present invention, designated ARMS, which contains a number of interesting features, including multiple ankyrin repeats, four putative transmembrane domains, a sterile alpha motif (SAM) domain, and a consensus PDZ-binding motif, is reported below. Interestingly, ARMS does not contain SH2 or PTB domains, suggesting it confers signaling specifity downstream of receptor tyrosine kinases in a manner distinct from classical adaptor proteins. An analysis of the structure and distribution of this protein suggests that it functions during development of the nervous system. Most important, ARMS is phosphorylated after treatment with NGF, BDNF and ephrin B2, indicating that it is a critical link between cell surface receptors and intracellular signaling events for both the neurotrophin and the ephrin families. The materials and methods used in the experiments in this example and the results obtained are provided below and discussed.

### MATERIALS AND METHODS

Materials

NGF was obtained from Harlan (Indianapolis, Ind.); BDNF from PeproTech (Rocky Hill, N.J.); NT4/5 was a generous gift of Regeneron (Tarrytown, N.Y.); EGF was obtained from Intergen (Purchase, N.Y.); and K252a from Calbiochem (La Jolla, Calif.).

Construction of Two-Hybrid DRG Library and Yeast Two-Hybrid Screen

A cDNA library was constructed into unique Bst XI/Not I sites of a modified version of pJG4-5. Briefly, polyadenylated RNA, which had been purified from adult mouse and P1 rat DRG using Trizol (Gibco, Gaithersburg, Md.) and the PolyA Tract System (Promega, Madison, Wis.), was used as template for reverse transcription (Gibco, Gaithersburg, MD) with an oligo-dT/Not I primer. Subsequent ligation into pJG4-5 and electroporation into DH5α yielded a library of approximately 10⁶ cfu and average insert size of 1.5-2.0 kilobases. A two-hybrid interaction screen, based on the LexA system (Gyuris et al., 1993), was performed in EGY48. The bait consisted of the entire cytoplasmic region of rat p75 as an in-frame fusion with the LexA DNA binding domain. Library cDNAs were expressed as in-frame fusions with the Gal4 transcriptional activation domain. Approximately 90 million yeast transformants were screened for their ability to survive in the absence of leucine. Sequence analysis identified a novel cDNA clone of approximately 2.5 kb that corresponded to the carboxy terminal 250 amino acids of ARMS.

Isolation of ARMS cDNA

Several rat brain libraries (young adult whole brain, adult whole brain and adult hippocampus) were screened to obtain the full-length ARMS cDNA. Criteria used to establish the initiator methionine include the following: assessment of multiple, independent cDNA fragments spanning the start site, an upstream, in-frame stop codon, and conformity to the Kozak consensus sequence.

Northern Blotting and in situ Hybridization

Total RNA was extracted from various rat tissues using Trizol reagent (Gibco, Gaithersburg, Md.). Twenty micrograms of total RNA were loaded per lane (with the exception of pancreas and DRG RNAs, which were <10 μg per lane), electrophoresed through a 2.2M formaldehyde/1% agarose gel, transferred to nylon membrane (Qiagen, Santa Clarita, Calif.), baked for 2 hours at 80° C., and probed with a cDNA fragment of ARMS labelled with ³²P-dCTP using Ready-To-Go (Amersham Pharmacia Biotech, Piscataway, N.J.). In situ hybridization was performed as previously described (Lai and Lemke, 1991). Briefly, 30 μm paraformaldehyde-fixed brain sections from adult rat or whole embryos were slide-mounted, hybridized with a ³²P-labelled cRNA probe generated by T7 RNA polymerase from a PCR-generated ARMS fragment, and washed at a final stringency of 0.1×SSC at 60° C. for 35 minutes. Emulsion-dipped slides were exposed for various times (days to weeks) prior to developing and then counterstained with thionin.

Cell Culture

Human embryonic kidney (HEK) 293 and 293T cells were maintained in Dulbecco's Modified Eagle's Medium (DMEM) containing 10% fetal bovine serum (FBS) supplemented with 100 U/ml penicillin, 100 μg/ml streptomycin, and 2 mM glutamine. Native PC12 cells, TrkA-overexpressing PC12 cells (615) (Hempstead et al., 1992), and TrkB-overexpressing PC12 cells were maintained in DMEM containing 10% FBS and 5% heat-inactivated horse serum with 30 U/ml penicillin, 30 μg/ml streptomycin, 2 mM glutamine, and 200 μg/ml G418.

SCG neurons were prepared from P2 rats and cultured on collagen-coated coverslips in C-medium (minimum essential medium containing 10% fetal bovine serum (FBS) supplemented with 0.4% glucose and 2 mM L-glutamine) with 150 ng/ml 2.5S NGF (Harlan, Indianapolis, Ind.). To inhibit growth of non-neuronal cells, neurons were cultured in the presence of 24.6 μg/ml 5-fluoro-2-deoxyuridine and 24.4 μg/ml uridine.

Dissociated primary cultures of hippocampal neurons from embryonic day 17 (E17) rats were prepared from timed-pregnant Sprague-Dawley rats following published procedures (Aibel et al., 1998). Following dissection of the hippocampus, the meninges were removed. The tissue was briefly minced with fine forceps and then triturated through a fire polished pasteur pipet. Cells were counted and plated in Neurobasal media supplemented with B27 (Gibco, Gaithersburg, Md.) on cell culture dishes coated overnight with 0.01 mg/ml poly-D-lysine. Cells were grown in a humidified incubator with 5% CO₂ at 37 C.

Antibodies

To characterize the expression of the ARMS protein, polyclonal antibody against the carboxy terminus of ARMS was generated. A bacterially expressed GST fusion protein with the carboxy-terminal 180 amino acids of ARMS was purified and used as antigen to generate rabbit antiserum (Cocalico Biologicals, Reamstown, Pa.). The specificity of this antiserum was determined by Western blot analyses of HEK293T cells transfected with a FLAG-tagged, full-length cDNA of ARMS. Lysates of transfected HEK293T cells, but not untransfected cells, displayed a specific 190 kilodalton species after immunoblotting with either an anti-FLAG antibody or anti-ARMS serum, 892.

Transfection of Mammalian Cells, Immunoprecipitation and Immunoblotting

For transient transfection experiments, HEK293 or HEK293T cells plated at 70-80% confluency in 10 cm dishes were subjected to calcium phosphate transfection with different combinations of the mammalian expression plasmids containing cDNAs for ARMS, rat TrkA, and rat HA-tagged p75 (Khursigara et al., 1999). PC12 615 cells or transiently transfected cells were harvested and lysed in 1 ml TNE buffer (10 mM Tris (pH 8.0), 150 mM NaCl, 1 mM EDTA, and 1% NP40) containing 0.12 mg/ml phenylmethylsulfonyl fluoride (PMSF), 2 μg/ml leupeptin, 1 μg/ml aprotinin, 10 mM sodium fluoride (NaF), and 1 mM sodium orthovanadate (Na₃VO₄) for 30 min on ice. Following centrifugation and removal of the insoluble fraction, protein concentration of the supernatant was determined by the Bio-Rad Protein Assay reagent (Bio-Rad Laboratories, Hercules, Calif.) with bovine serum albumin as the standard. Cell lysates of equivalent protein content were incubated for 4 hours to overnight with rotation at 4° C. with either anti-pan-Trk polyclonal antibody, C14 (1.5 μg) or anti-ARMS (892) antiserum (1:100). The immune complexes were immobilized on protein A-Sepharose beads (Sigma), washed six times with ice-cold TNE buffer, boiled in SDS-sample buffer, separated by sodium dodecyl sulfate-polyacrylamide gel electrophoresis (SDS-PAGE), and transferred onto PVDF membrane (Millipore, Bedford, Mass.). Immunoblotting was carried out by first blocking membranes in TBST buffer (20 mM Tris (pH 7.5), 500 mM NaCl, 0.1% Tween-20) containing 5% BSA for pY99 and 5% nonfat milk for others, and then incubating for 2 hours at room temperature or overnight at 4° C. in TBST buffer containing one of the following primary antibodies: anti-Trk 44 serum (1:2000); anti-phosphotyrosine pY99 antibody (0.1 μg/ml) (Santa Cruz Biotechnology, Santa Cruz, Calif.); anti-ARMS (892) antiserum (1:1000). Membranes were washed with TBST buffer and then incubated with horseradish peroxidase (HRP)-conjugated goat anti-rabbit (Roche Molecular Biochemicals, Indianapolis, Ind.) or goat anti-mouse secondary antibodies (Jackson ImmunoResearch Laboratories, West Grove, Pa.) at 1:10,000 and 1:7500 dilutions, respectively. Immunoreactive protein bands were detected by enhanced chemiluminescence using ECL reagents (Amersham Pharmacia Biotech, Piscataway, N.J.).

Immunofluorescence Analysis

SCG neurons were fixed with 4% paraformaldehyde and blocked with PBS containing 0.075% saponin and 10% FBS or normal goat serum (NGS). Cells were then incubated with anti-ARMS (892, 1:1000) or anti-Trk B-3 antibody (1 μg/ml) in blocking buffer. Primary antibodies were visualized using fluorescence-conjugated secondary antibodies (Cy3-conjugated goat anti-mouse IgG (Jackson Lab; 1:200) or FITC-conjugated goat anti-rabbit IgG (Jackson Lab; 1:100)). Images were collected on a BioRad confocal microscope.

### RESULTS

Cloning of Ankyrin Repeat-Rich, Membrane Spanning (ARMS)

Neurotrophins exert many biological activities, but few signaling molecules have been found to be specific for neurotrophin receptor signaling. The majority of proteins that serve as substrates for neurotrophin receptors are utilized by other receptor families. Effects on cellular and axonal migration, nerve regeneration, and apoptosis have been implicated for the p75 neurotrophin receptor, and effects on long term potentiation and synaptic transmission have been attributed to the Trks. To define unique molecules in neurotrophin signaling, a two-hybrid screen was undertaken using a rat dorsal root ganglion library as prey and the cytoplasmic domain of p75 as bait. This report describes a p75-interacting protein that is a downstream target for Trk signaling.

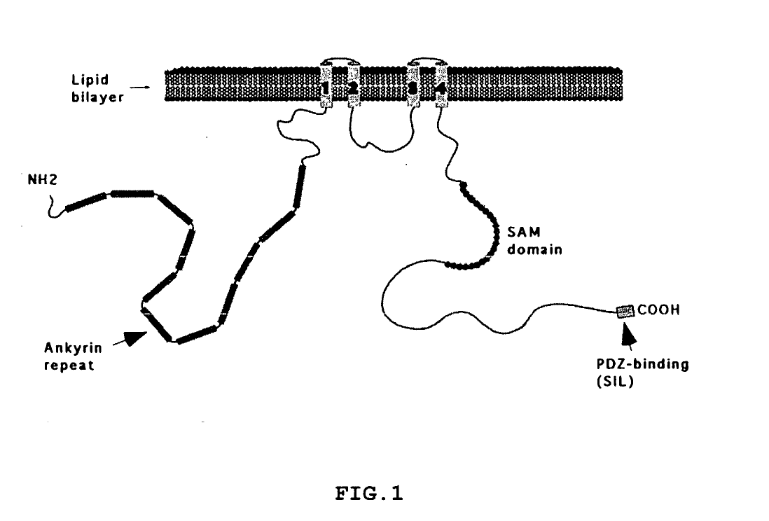

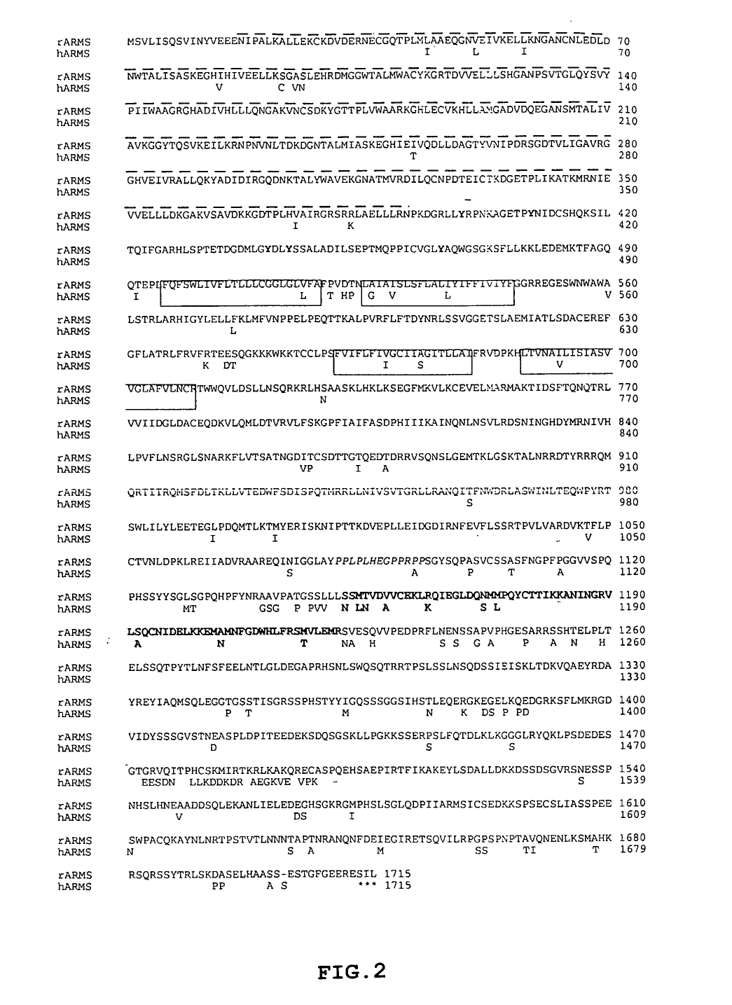

A positive cDNA clone was identified that encoded the carboxy terminal portion of a novel 1,715 amino acid protein. The predicted amino acid sequence contained eleven contiguous, 33 amino acid ankyrin repeats in the N-terminal domain and four putative membrane-spanning regions. This protein was designated as ARMS for Ankyrin Repeat-rich, Membrane Spanning. The overall topology of ARMS is decidedly different from other transmembrane proteins. Other distinguishing features of this protein include a SAM domain, a polyproline stretch, and a potential PDZ-binding motif (FIGS. 1 and 2). These motifs represent candidate protein-protein interaction domains.

An extensive search of GenBank databases for proteins similar to ARMS identified many ankyrin-containing proteins; however, proteins sharing homology with ARMS in regions outside of the ankyrin repeats were notably absent. Analysis of C. elegans and Drosophila databases revealed ARMS orthologs in these organisms. Further analysis of human databases allowed the present inventors to determine the sequence of human ARMS from two overlapping ESTs in the database. The presence and conservation of ARMS sequences from nematodes to humans suggests that this protein may serve evolutionarily conserved functions. Between rat and human, the amino acid identity is 91% and similarity is 94% (FIG. 2). Comparison of ARMS sequences among human, rat, Drosophila and C. elegans revealed similarity in overall structure: multiple ankyrin repeats in the amino terminus, four putative transmembrane domains, SAM domains, and consensus PDZ-binding motifs at the carboxymost three amino acids (“SIL” in human and rat; “TKL” in Drosophila, and “SDA” in C. elegans). Despite the relatively divergent carboxy termini among the ARMS homologs, the presence of potential PDZ recognition sequences in all of these proteins indicates that conserved protein-protein interactions for ARMS may exist.

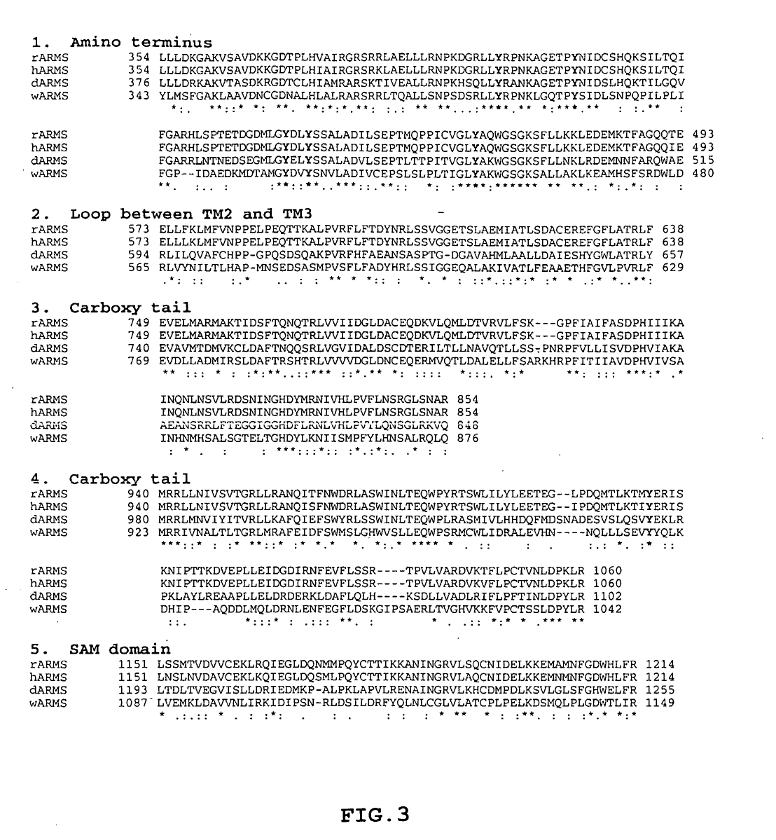

Interestingly, the regions most highly conserved were not the transmembrane (TM) domains, but certain regions surrounding them, namely the ankyrin repeats, the amino terminal portion between the ankyrin repeats and the first TM domain, the “loop” region between TM domains 2 and 3, and several stretches of amino acids in the carboxy terminus downstream of TM 4, including the SAM domain (FIG. 3). The regions highlighted in FIG. 3 range between 20% to 42% identical and 56% to 77% similar, whereas the TM domains are only 1% identical and 43% similar. It is interesting to note that embedded in these subdomains are conserved tyrosine residues; these potential phosphorylation sites are conserved among all four organisms examined (FIG. 3, part 1). Although mutations in C. elegans and Drosophila ARMS have not yet been described, the C. elegans homolog, F36H1.2, is most similar to the ankyrin-related gene, UNC-44 (28%), which has a role in axon guidance, axonogenesis, and neuronal development (Otsuka et al., 1995).

Distribution of ARMS Transcripts in Rat

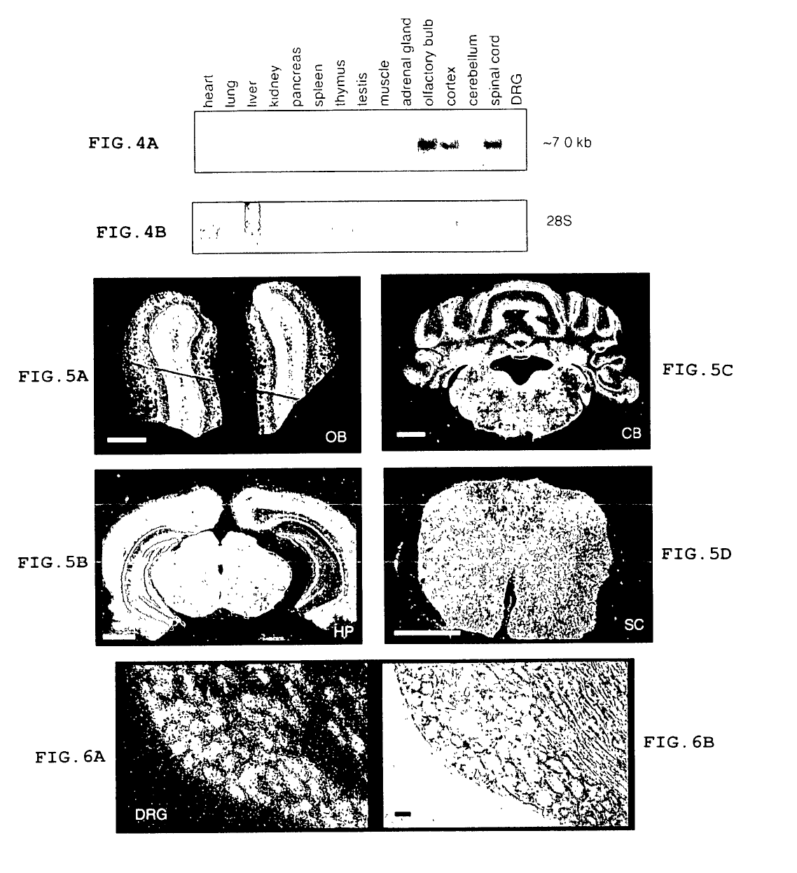

In rat tissue, expression of ARMS mRNA was assessed by Northern blot analysis using a probe directed against the membrane-spanning regions of ARMS (FIG. 4A). A single transcript of approximately 7.0 kilobases was detected. Although ARMS mRNA could be detected in several non-neuronal tissues, it was most abundant in the nervous system.

The expression of ARMS by in situ hybridization in adult rat brain was examined. Several populations of neurons were found to express ARMS mRNA, including mitral cells and cells of the glomerular layer of the olfactory bulb, all regions of the hippocampus, Purkinje cells of the cerebellum, and grey matter (most notably in presumed large motor neurons) of the spinal cord (FIGS. 5A-5D). One shared property of these neuronal populations is their ability to undergo synaptic changes throughout adulthood.

During defined periods of rodent embryonic development, subpopulations of sensory neurons require distinct neurotrophins and their cognate receptors for survival (Pinon et al., 1996). The neurotrophin receptor system remains functional through adulthood. Correspondingly, ARMS expression coincides with that of the Trks and p75. As shown by Northern analysis (FIG. 4A) and in situ hybridization (FIGS. 6A-6B), ARMS expression persists in the adult DRG. In addition to an absence of silver grains over the nerve fibers, there was a notable absence of ARMS message in a subset of DRG cell bodies corresponding to large diameter neurons. Although ARMS mRNA can be detected in all populations of DRG neurons, much lower expression in large diameter neurons suggests a less prominent role in proprioception.

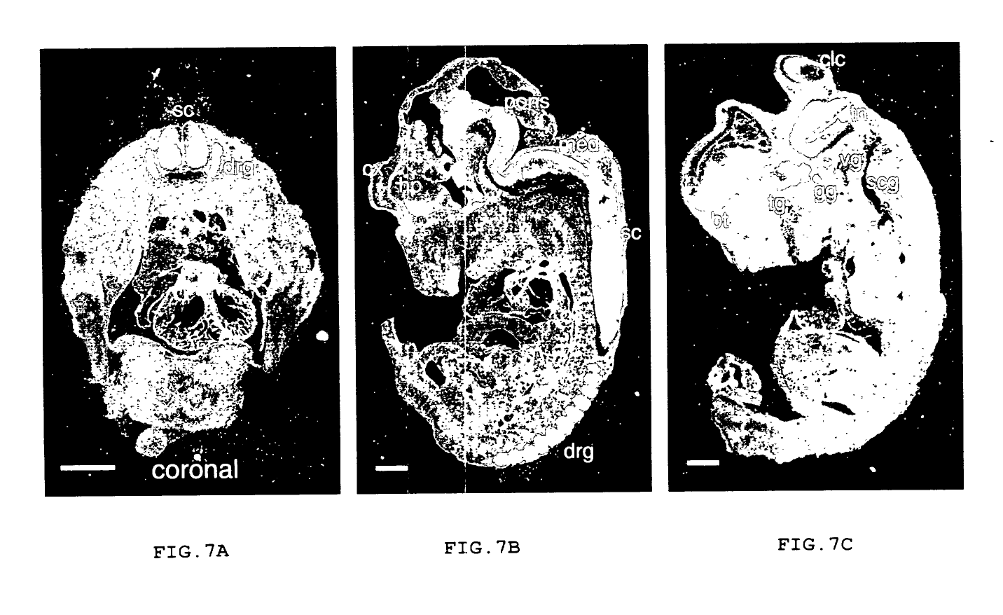

More striking was the restricted expression of ARMS during development. In general, during rat nervous system development, the period of embryonic growth between E11 to E14 is a time of massive proliferation of the neuroepithelium. By E14, the first sets of postmitotic neurons are undergoing differentiation. In E14 rat embryos, ARMS was expressed in both spinal cord and dorsal root ganglia (FIG. 7A). These neuronal populations are among the first in the nervous system to undergo differentiation, and it is during this window of development that these postmitotic neurons are actively seeking and making connection with their targets. Additionally, several neuroanatomical loci of the developing brain expressed ARMS mRNA. In the midsagittal plane, the hippocampus, cortex, pons, and medulla were positive for ARMS expression (FIG. 7B). Regions lateral to the midline such as the basal telencephalon, superior/inferior colliculus, principal trigeminal sensory nucleus, and multiple ganglia, including trigeminal, geniculate, vestibular, and superior cervical (FIG. 7C) showed significant levels of ARMS transcripts.

Association of ARMS with NGF Receptors

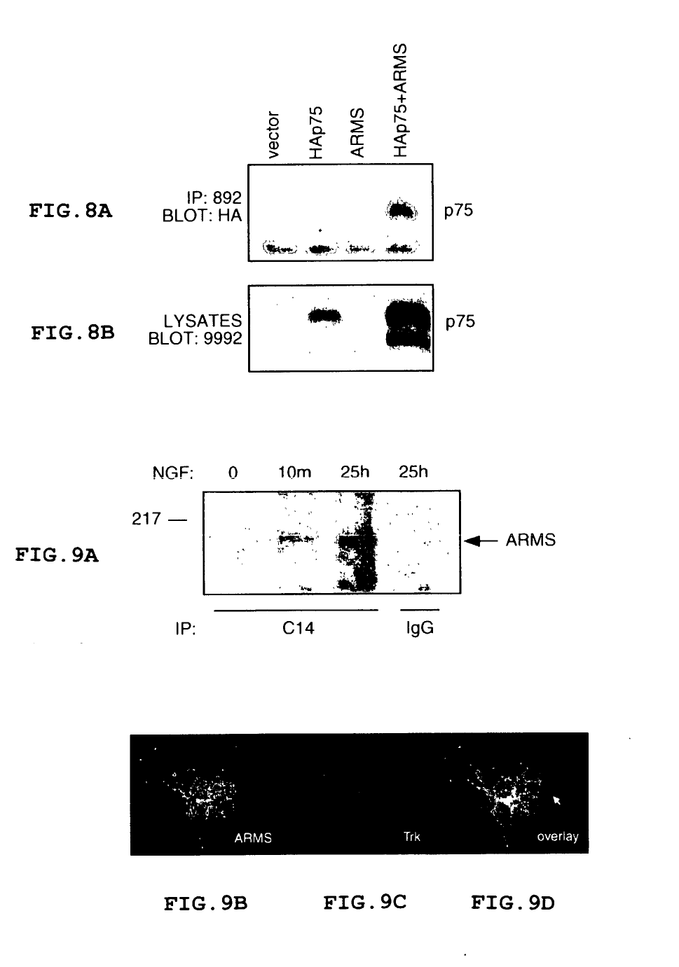

Since the ARMS protein was originally identified in a yeast two-hybrid assay from its association with the cytoplasmic domain of the p75 receptor, the laboratory of the present inventors tested whether ARMS was capable of an interaction with p75 in cultured cells. HA-tagged p75 cDNA and the ARMS cDNA were co-transfected in HEK293T cells. After immunoprecipitation with an antibody (892) made against the C-terminal region of the ARMS protein, the p75 receptor could be readily detected by Western analysis (FIGS. 8A and 8B). This transfection experiment indicated that ARMS and p75 are capable of forming a complex in HEK293T cells.

Previous studies indicated that p75 and the TrkA receptor can exist in a complex (Huber and Chao, 1995; Gargano et al., 1997; Bibel et al., 1999). To establish whether an interaction between TrkA and ARMS occurs, an immunoprecipitation experiment in PC12 cells expressing elevated levels of TrkA was carried out (Hempstead et al., 1992). An NGF-dependent association between ARMS and the TrkA receptor was detected following immunoprecipitation of TrkA and immunoblotting with the anti-ARMS antibody, 892 (FIG. 9A). The association between ARMS and TrkA persisted 25 hours after NGF treatment. The endogenous association of TrkA and ARMS indicates that ARMS may exist in a ligand-dependent complex with Trk receptors.

To investigate further the distribution and potential co-localization of ARMS and Trk in neuronal cells, indirect immunofluorescence experiments in primary cultures of rat sympathetic neurons. Using the 892 antibody against ARMS (FIG. 9B) and a monoclonal antibody directed against Trk (FIG. 9C), co-localization of ARMS and TrkA on the cell surface of sympathetic neurons was observed (FIG. 9D, arrow). These results support the co-immunoprecipitation studies that suggest a physical association between Trk and ARMS.

Tyrosine Phosphorylation of ARMS by NGF

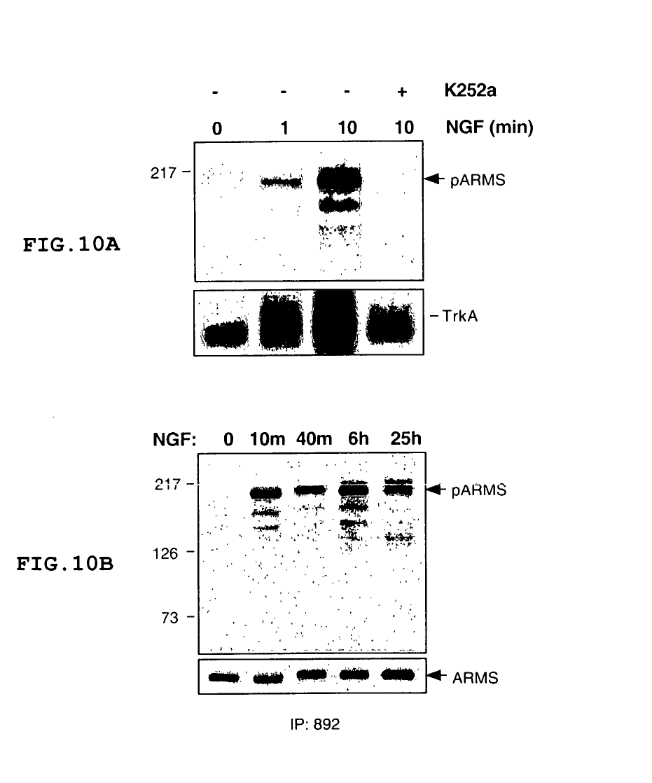

The high degree of correspondence in the expression of ARMS and neurotrophin receptors and the endogenous association with TrkA receptors led the present inventors to postulate that ARMS might function as a target for Trk receptor phosphorylation. To investigate this possibility, cell lysates from NGF-treated PC12 cells were prepared and the ARMS protein was immunoprecipitated with anti-ARMS antiserum, 892. Phosphorylation of ARMS protein was visualized by immunoblotting with an anti-phosphotyrosine antibody, pY99. Tyrosine phosphorylation of ARMS was detectable within a minute of NGF addition (FIG. 10A, top), closely following the time course of TrkA autophosphorylation (FIG. 10A, bottom). With continued NGF treatment, the phosphorylation of ARMS persisted for 25 hours (FIG. 10B). Pretreatment with the alkaloid-like compound K-252a (100 nM), which specifically inhibits NGF-mediated activity by selectively blocking the kinase activity of TrkA (Koizumi et al., 1988; Berg et al., 1992), completely abolished the tyrosine phosphorylation of ARMS. Hence, the ARMS protein represents a novel downstream target for the TrkA receptor tyrosine kinase.

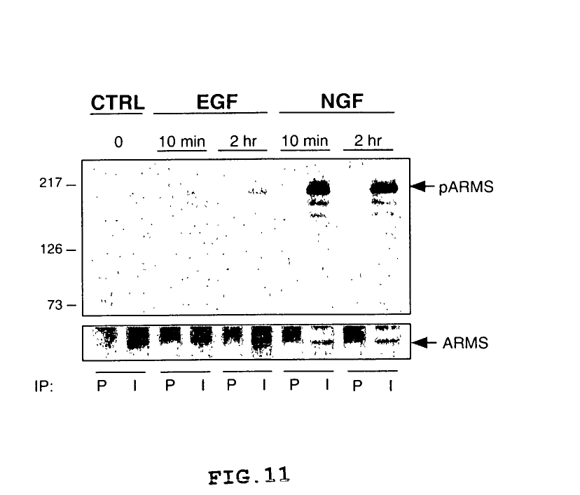

To explore the specificity of ARMS phosphorylation, the effects of EGF and NGF were compared in PC12 cells. The phosphorylation of ARMS was considerably more robust following NGF treatment (FIG. 11), even though both TrkA and EGF receptors were activated. These data indicate that ARMS phosphorylation by TrkA was a specific consequence of NGF, but not EGF, receptor signaling. This may be a consequence of a lack of association of ARMS with the EGF receptor. It is unlikely that ARMS phosphorylation was mediated through the MAP kinase pathway, since treatment of PC12 cells with EGF showed minimal tyrosine phosphorylation of ARMS after 10 minutes or 2 hours (FIG. 11). Also, the pronounced ARMS phosphorylation was not elicited by other growth factors, such as insulin, FGF and insulin growth factor-1, in PC12 cells (data not shown), indicating unique proteins are phosphorylated by the receptor tyrosine kinase, TrkA.

Response to Other Trophic Factors and Ephrins

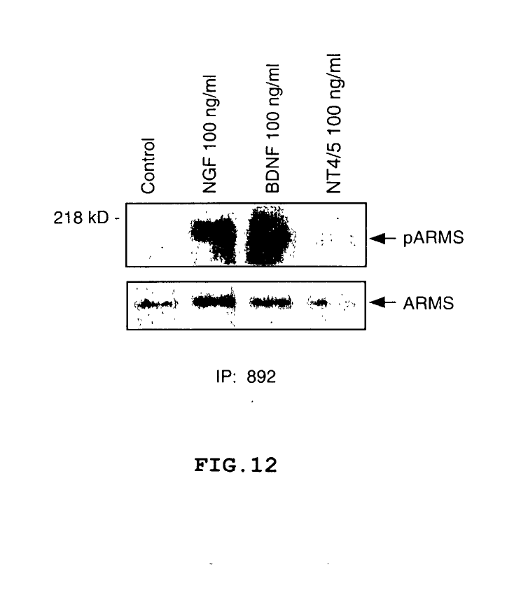

To test the ability of other Trk receptors to phosphorylate ARMS, PC12 cells stably expressing the TrkB receptor were used. Upon treatment of these cells with BDNF (100 ng/ml), ARMS was tyrosine phosphorylated, demonstrating that ARMS can be phosphorylated by TrkB as well as TrkA (FIG. 12). That expression of ARMS is localized to some central regions in which TrkA is not expressed, such as the hippocampus, can be reconciled by the presence of TrkB, which is widely expressed in the adult CNS and which serves as a receptor for BDNF and NT-4/5 (Farinas et al., 1998).

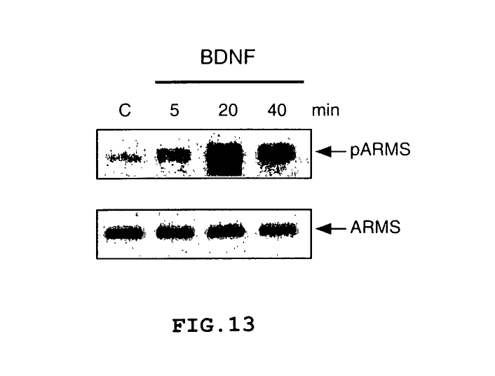

To verify the ability of BDNF to induce phosphorylation of ARMS in neurons, primary cultures of hippocampal neurons were established. Hippocampal neurons express the TrkB receptor. After treatment with BDNF, ARMS was immunoprecipitated from hippocampal lysates and assessed for tyrosine phosphorylation by immunoblotting with an anti-phosphotyrosine antibody. A similar phosphorylation of ARMS after BDNF treatment in hippocampal neurons was observed (FIG. 13). Thus, in a primary neuronal culture, ARMS is a downstream target of TrkB after activation by BDNF.

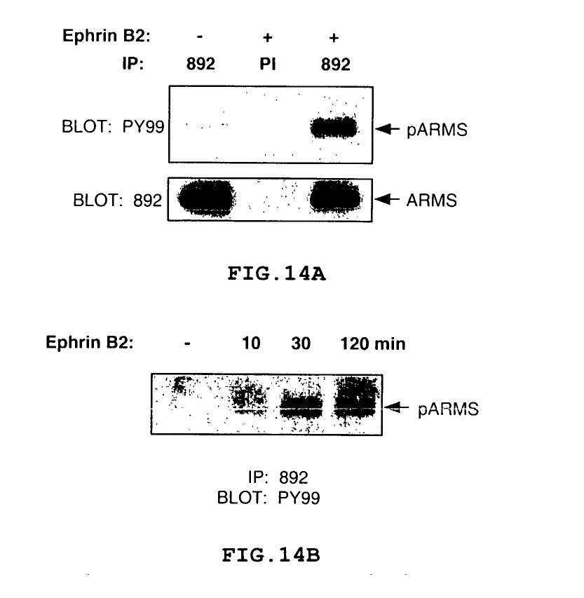

The expression of ARMS during developmental periods of axon outgrowth and synaptogenesis raises the possibility that ARMS is downstream of other known axon guidance regulators. The ephrin family was therefore examined because of its well-established in vitro and in vivo guidance function. As seen in FIG. 14A, the ARMS protein is potently tyrosine phosphorylated by ephrin B2 in a neuronal/glioma hybridoma cell line, NG108-15, that stably expresses EphB2 receptor. EphB2 receptor autophosphorylation and activation occurs over a time period of 30-40 minutes (Holland et al., 1997), in contrast to a much more rapid activation for Trks. Significantly, the phosphorylation of ARMS followed a time course similar to the time course of Eph receptor autophosphorylation (FIG. 14B).

Unlike the Trk receptors, which are activated upon dimerization by their respective ligands, Eph receptors are aggregated into multimeric complexes to be biologically active. This differential property of receptor aggregation may account for the differential time courses observed between neurotrophins and ephrins. Interestingly, the time course of TrkA and EphB2 phosphorylation of ARMS suggests that ARMS may be phosphorylated directly by the kinase domains of the receptors themselves and not via downstream intermediates. The integral membrane nature of ARMS and its potentially close proximity to receptor tyrosine kinases could facilitate such a process. Most important, these results suggest that ARMS is phosphorylated by receptor tyrosine kinases with established roles in axonal targeting and guidance. It remains to be determined whether ephrin-Eph clustering and subsequent ARMS phosphorylation is involved in attractive or repulsive effects during axon guidance.

### DISCUSSION

ARMS: A Novel Downstream Target for Receptor Tyrosine Kinases

Although the signaling properties of the Trk receptor tyrosine kinases have been studied extensively, there remain many neurotrophin-stimulated activities in which molecular mechanisms have not been fully defined. These include internalization and transport of receptors, growth cone guidance, and axonal and dendritic branching. A number of common substrates, including phospholipase C-γ, PI-3 kinase, and Shc and Grb2 adaptor proteins, are utilized by many receptor tyrosine kinases, raising the question of how phosphorylation events lead to different biological outcomes (Chao, 1992b). One possibility is that there remain unique substrates that determine the specific nature of neurotrophin responses.

Here, the properties of a novel tyrosine phosphorylated transmembrane protein are described. The ARMS protein is highly expressed in many neuronal populations and functions as a downstream target for both Trks and Eph receptor. A common feature of ARMS, p75, TrkA, and Eph receptors is that they all contain C-terminal PDZ-binding motifs. It is therefore possible that these proteins are localized to the same subcellular compartment by a PDZ-containing molecule, and this localization may contribute to signaling events both developmentally and in adulthood. Indeed, the EphB2 receptor has been found to be associated with PDZ-containing proteins, and this association is thought to be important for receptor function at neuronal synapses (Torres et al., 1998) and for vestibular axon guidance and ionic homeostasis in the inner ear (Cowan et al., 2000).

At E14, trigeminal and vestibular axons migrate toward their peripheral targets and commence synapse formation. High levels of ARMS mRNA expression in these subpopulations suggest that ARMS may participate in axonogenesis or axon guidance. A notable absence of ARMS mRNA expression was observed in germinal zones of the developing brain, regions that are extensively proliferating. A general conclusion that can be drawn from the in situ hybridization studies described here is that ARMS-positive neuron populations, for the most part, are post-mitotic and post-migratory. For example, the somatic and visceral motor neurons of the spinal cord are born by E12 and E13, but by E14 are already undergoing axonogenesis (Paxinos, 1995). It is during this post-migratory, differentiative stage that ARMS is highly expressed. Conversely, the presumptive olfactory bulb, which is devoid of neurons at this stage of development, is negative for ARMS expression; the absence of ARMS expression in pre-migratory neuronal populations supports the observation that ARMS expression may be restricted to post-mitotic mitotic and post-migratory neurons. These findings suggest that ARMS may be involved in post-migratory events, such as axon guidance or synaptogenesis.

Expression of ARMS in the adult central nervous system was observed in regions such as the olfactory bulb, hippocampus, Purkinje cells of the cerebellum, and spinal cord motor neurons. The most significant commonality of these neuronal populations is their ability to undergo continued synaptic changes throughout adult life. Neurons of the olfactory bulb are continually renewed and hence must form new synapses; hippocampal neurons can undergo synaptic remodeling and LTP; Purkinje cell dendrites are highly plastic due to their constant structural remodeling; and motor neurons have the capacity to regenerate and to form new synapses with peripheral targets in adults. Peripheral neurons have regenerative properties throughout adulthood, and this process requires axon outgrowth and new synapse formation. Neurotrophins and ephrins are likely candidates in spinal cord regeneration (Frisen et al., 1992; Miranda et al., 1999), and the present inventors believe that ARMS is also utilized in this process.

Colocalization of ARMS with Neurotrophin and Ephrin Receptors

The in vivo utilization of ARMS by the neurotrophin and ephrin receptors is supported by co-expression of these proteins during development. For example, Trk and p75 expression can be detected in all peripheral ganglia of neural crest origin (e.g., superior cervical and dorsal root), spinal cord, and brainstem, regions also high in ARMS mRNA (Yan and Johnson, 1988; Snider, 1994). In the adult, ARMS and p75 expression overlap most notably in the olfactory bulb, Purkinje cells of the cerebellum and motor neurons of the spinal cord. Additionally, Trk receptors are found throughout the nervous system in regions that also express ARMS. For example, TrkA and ARMS are both expressed in the small diameter sensory neurons of the DRG (Averill et al., 1995; Wright and Snider, 1995). Adult hippocampus, cerebellar Purkinje cells, and motor neurons of the spinal cord are a few regions in which the expression of ARMS and TrkB significantly overlaps (Yan et al., 1997).

In a similar manner, Eph receptor and ARMS expression overlap in adult hippocampus and spinal motor neurons. During development, the Eph receptors are also found in ganglia of neural crest origin (e.g., sensory and vestibular) and in the tectum (Flanagan and Vanderhaeghen, 1998), regions that also express ARMS. Ephrins and their receptors have a well known function in establishment of the retinotectal pathway, and the expression of ARMS in the tectum suggests that ARMS plays a role in this process.

Given the multiple cell populations that express Trks, Eph receptors and ARMS, such as sensory and motor neurons, our findings raise the possibility of cross-talk between these two receptor systems. ARMS may serve to link or modulate these two signaling pathways in a cooperative or competitive manner. This could potentially be achieved by receptor phosphorylation of similar or different tyrosine residues of ARMS and/or association with other membrane proteins. It is plausible that ARMS may serve as an adaptor protein, given its PDZ-binding motif and other protein interaction domains that would allow for recruitment of multiple, diverse proteins.

### EXAMPLE 2

### MATERIALS AND METHODS

Yeast Two-Hybrid Screening

The two hybrid interaction screenings were performed using 3 different baits named 2.1, 7 and 10 that corresponded to amino acids S1303-L1715, D1056-S1151 and L1603-L1715 of ARMS protein (SEQ ID NO:2), respectively. The bait plasmids were generated by PCR with rat ARMS cDNA and the amplified fragments were ligated into pEG202 at BamHI-NcoI sites as an in-frame fusion with the LexA-DNA binding domain.

A cDNA library from postnatal day (P1) dorsal root ganglia (DRG) was generated (see Example 1). The DRG library cDNAs were expressed as in-frame fusions with the Gal4 transcriptional activation domain. Approximately 5×10⁷ yeast transformants were screened for growth in absence of leucine. Positive-interacting clones were confirmed with β-galactosidase activity and their specificity was tested using unrelated proteins as baits.

### RESULTS

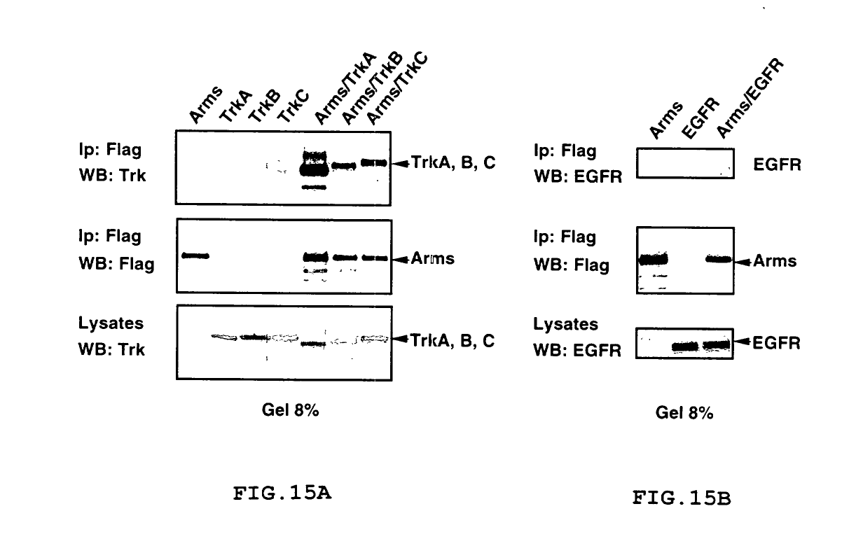

As demonstrated in Example 1, FIGS. 15A and 15B show that ARMS interacts with TrkA, TrkB, and TrkC receptors but not with EGF receptors.

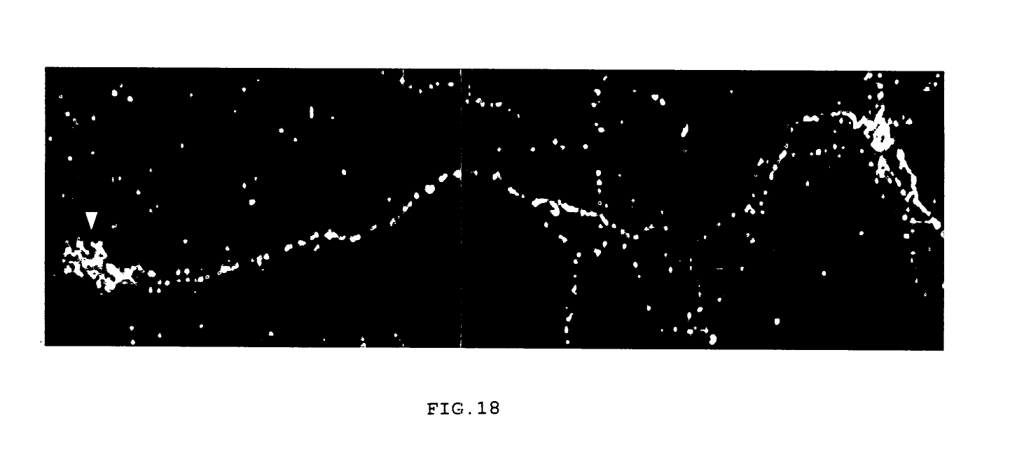

The laboratory of the present inventors have also found that following phosphorylation, ARMS becomes localized discretely at growth cones and in synaptic regions of neuronal membranes as visualized using anti-ARMS antibodies in immunofluorescence studies. FIG. 18 is an immunofluorescence analysis that shows ARMS localized in growth cones and axons of hippocampal neurons. It was further demonstrated that ARMS co-localizes with Vamp-2, a synaptic vesicle marker, in hippocampal neurons (FIGS. 17A-17F).

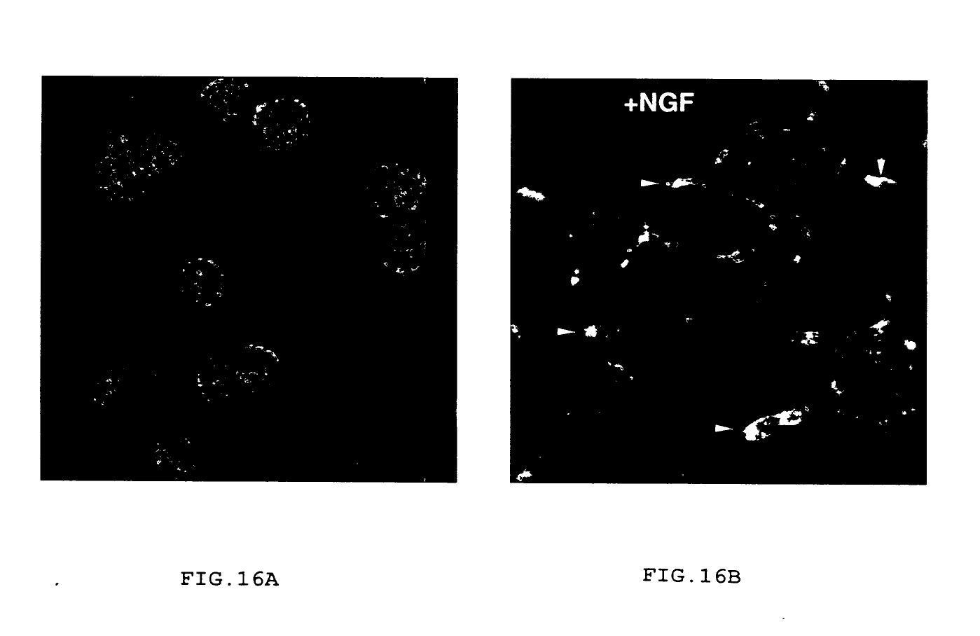

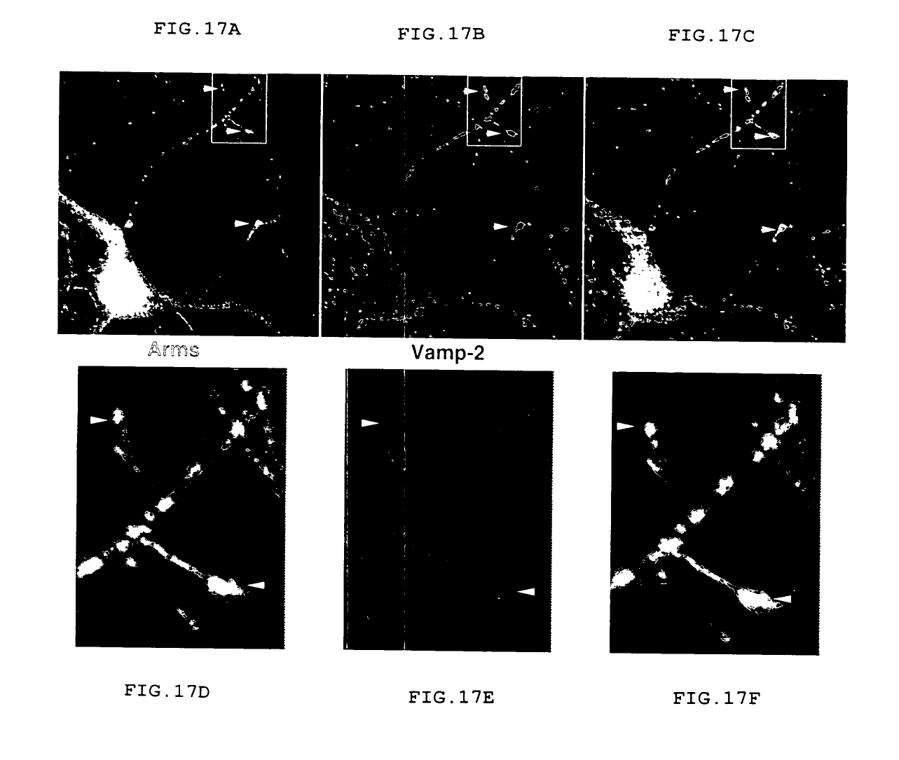

The results of immunofluorescence and confocal microscopic studies show that there is a dramatic change in distribution of ARMS in both hippocampal neurons and PC12 cells after treatment with BDNF or NGF (FIGS. 16A and 16B), indicating that ARMS becomes concentrated at synaptic sites where neuronal activities are located.

The laboratory of the present inventors further identified several neuronal proteins (Table 1) that interact/bind with the ARMS protein using a yeast two-hybrid screening system.

TABLE 1
Proteins Interacting with ARMS

| PROTEIN | LOCALIZATION AND FUNCTION | REFERENCES |
|---|---|---|
| Cell division control | Predominantly expressed in the brain and | Beites et al., 1999 |
| related Protein 1 | associates with membranes incluing synaptic | |
| (CDCrel-1) | vesicles. | |
| | Inhibition of exocytosis | |
| Calcium channel beta-3 | Predominantly expressed in the brain but also in | Castellano et al., 1993 |
| subunit | aorta, lung, heart, pancreas and the adrenal gland. | |
| | Regulation of Ca²⁺ entry into the cell | |
| Actin related protein ⅔ | Part of a complex implicated in the control of | Welch et al., 1997 |
| complex, subunit 3 (21 | actin polymerization in cells. | |
| kDa) (ARPC3) | | |
| Synembryn | Regulation of neurotransmitter secretion by | Miller et al., 2000 |
| | controlling the production and consumption of | |
| | diacylglycerol. | |
| GRIP1 and GRIP2 (ABP) | enriched in brain synapses | Dong et al., 1997 |
| | Possible role in either an anchoring of AMPA | Srivastava et al., 1998 |
| | receptors at synapses or in the trafficking of | |
| | AMPA receptors | |

The proteins in Table 1, identified as interacting with ARMS, include several synaptic vesicle proteins, a calcium channel subunit and proteins involved in clustering glutamate channels (NMDA, kainate). These interactions suggest that the ARMS protein is involved in neurotransmitter vesicle movements in nerve cells, in the release of neurotransmitters, i.e., acethylcholine or dopamine, and in the clustering of ion channels, all of which cause changes in neuronal activity (action potentials).

There is growing evidence that neurotrophins, such as NGF and BDNF, affect neurotransmitter release and neuronal activity, in addition to promoting neuronal survival (Auld et al., 2001; Albeck et al., 1999; Pozzo-Miller et al., 1999; Jia et al., 1999; Sala et al., 1998; Kang et al., 1995; Korte et al., 1995; Toledo-Aral et al., 1995). The significance of these findings stem from the potent effects of neurotrophins upon synaptic plasticity. The phosphorylation of ARMS by Trk and Eph receptors provides a mechanism for transmitting information from neurotrophins to specific regions of synaptic activity in nerve cells.

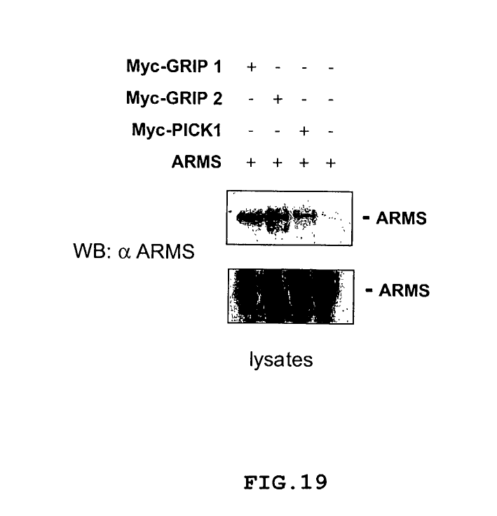

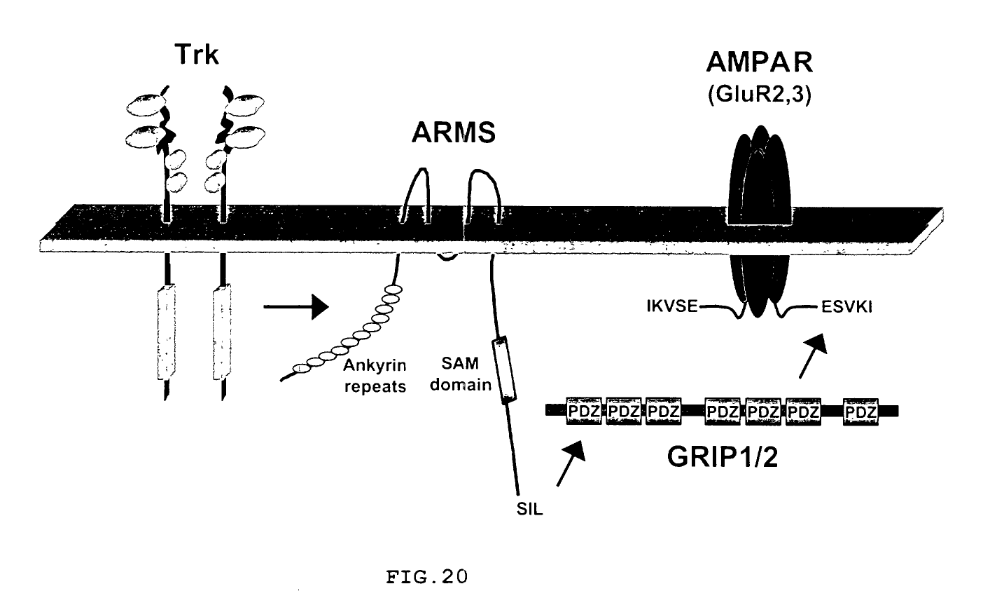

PDZ proteins, such as GRIP1 and GRIP2 in Table, contain a protein interaction domain (PDZ) that bind in a sequence specific way to short C-terminal sequences in a wide variety of membrane proteins. The function of PDZ proteins is to assemble complexes of proteins that perform signaling functions in specific subcellular locations, such as at synapses or growth cones. PDZ-containing proteins have been shown to cluster AMPA receptors, such as GluR2 and GluR3 (Dong et al 1997; Srivastava et al 1998). The Trk receptors were demonstrated to associate with ARMS (Example 1 and FIGS. 15A and 15B). Furthermore, ARMS can recruit PDZ proteins such as GRIP1 and GRIP2 (FIG. 19), which possesses seven PDZ motifs. This interaction between ARMS and GRIP proteins is most likely between the C-terminus of ARMS (SIL) and the first PDZ repeat of GRIP. The AMPA glutamate receptors bind to the 7^{th} PDZ repeat of GRIP through its C-terminal sequence VKI and can also bind to the 3^{rd}-6^{th} DZ repeats of GRIP.

These binding interactions have been extensively defined for many proteins (Sheng et al., 2001). The interactions between ARMS and GRIP proteins link ARMS to ion channels which can regulate neuronal activity. Since ARMS is a specific and robust substrate for Trk receptor phosphorylation, neurotrophins may therefore communicate with components of synapses through the ARMS and PDZ-containing proteins. This provides a potential mechanism to explain changes in synaptic plasticity that have been detected with neurotrophins (Thoenen, 1995 Poo, 2001).

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### REFERENCES

Aibel L, Martin-Zanca D, Perez P, Chao M V (1998) Functional expression of TrkA receptors in hippocampal neurons. J Neurosci Res 54:424-431.

Albeck D S, Backman C, Veng L, Friden, P et al. Acute application of NGF increases the firing rate of aged rat basal forebrain neurons. Eur J Neurosci 11, 2291 (1999)

Auld D S, Mennicken F and Quirion R. Nerve growth factor rapidly induces prolonged acetylcholine release from cultured basal forebrain neurons. J Neuroscience 21, 3375 (2001)

Averill S, McMahon S B, Clary D O, Reichardt L F, Priestley J V (1995) Immunocytochemical localization of trkA receptors in chemically identified subgroups of adult rat sensory neurons. Eur J Neurosci 7:1484-1494.

Barker P A, Shooter E M (1994) Disruption of NGF binding to the low affinity neurotrophin receptor p75LNTR reduces NGF binding to TrkA on PC12 cells. Brain Res 664:155-166.

Beites, C L, Xie H, Bower R, and Trimble W S. (1999) The septin CDCrel-1 binds syntaxin and inhibits exocytosis. Nat Neurosci 2, 434-439.

Benedetti M, Levi A, Chao M V (1994) Differential expression of nerve growth factor receptors leads to altered binding affinity and neurotrophin responsiveness. J Neurosci 14:29-38.

Berg M M, Sternberg D W, Parada L F, Chao M V (1992) K-252a inhibits nerve growth factor-induced trk proto-oncogene tyrosine phosphorylation and kinase activity. J Biol Chem 267:13-16.

Bibel M, Hoppe E, Barde Y A (1999) Biochemical and functional interactions between the neurotrophin receptors trk and p75NTR. Development 126:2129-2140.

Bonhoeffer T (1996) Neurotrophins and activity-dependent development of the neocortex. Curr Opin Neurobiol 6:119-126.

Brennan C, Rivas-Plata K, Landis S C (1999) The p75 neurotrophin receptor influences NT-3 responsiveness of sympathetic neurons in vivo. Nat Neurosci 2:699-705.

Bruckner K, Klein R (1998) Signaling by Eph receptors and their ephrin ligands. Curr Opin Neurobiol 8:375-382.

Castellano A, Wei X, Birnbaumer L, and Perez-Reyes E. (1993) Cloning and expression of a third calcium channel beta subunit. J Biol Chem 268, 3450-3455.

Castellano A, Wei X, Birnbaumer L, and Perez-Reyes E. (1993) Cloning and expression of a neuronal calcium channel beta subunit. J Biol Chem 268, 12359-12366.

Caterina M J, Schumacher M A, Tominaga M, Rosen T A, Levine J D, Julius D (1997) The capsaicin receptor: a heat-activated ion channel in the pain pathway [see comments]. Nature 389:816-824.

Chao M V (1992a) Growth factor signaling: where is the specificity? Cell 68:995-997.

Chao M V (1992b) Neurotrophin receptors: a window into neuronal differentiation. Neuron 9:583-593.

Cowan C A, Yokoyama N, Bianchi L M, Henkemeyer M, Fritzsch B (2000) EphB2 guides axons at the midline and is necessary for normal vestibular function. Neuron 26:417-430.

Dong H, O'Brien R J, Fung E T, Lanahan A A, Worley P F, and Huganir R L. (1997) GRIP: a synaptic PDZ domain-containing protein that interacts with AMPA receptors. Nature 386, 279-284

Eshhar et al., (1990) Chimeric T cell receptor which incorporates the anti-tumour specificity of a monoclonal antibody with the cytolytic activity of T cells: a model system for immunotherapeutical approachBr. J. Cancer Suppl., 10:27-9

Farinas I, Wilkinson G A, Backus C, Reichardt L F, Patapoutian A (1998) Characterization of neurotrophin and Trk receptor functions in developing sensory ganglia: direct NT-3 activation of TrkB neurons in vivo. Neuron 21:325-334.

Flanagan J G, Vanderhaeghen P (1998) The ephrins and Eph receptors in neural development. Annu Rev Neurosci 21:309-345.

Frisen J, Holmberg J, Barbacid M (1999) Ephrins and their Eph receptors: multitalented directors of embryonic development. Embo J 18:5159-5165.

Frisen J, Verge V M, Cullheim S, Persson H, Fried K, Middlemas D S, Hunter T, Hokfelt T, Risling M (1992) Increased levels of trkB mRNA and trkB protein-like immunoreactivity in the injured rat and cat spinal cord. Proc Natl Acad Sci USA 89:11282-11286.

Gallo G, Lefcort F B, Letourneau P C (1997) The trkA receptor mediates growth cone turning toward a localized source of nerve growth factor. J Neurosci 17:5445-5454.

Gargano N, Levi A, Alema S (1997) Modulation of nerve growth factor internalization by direct interaction between p75 and TrkA receptors. J Neurosci Res 50:1-12.

Gross et al., (1989) Expression of immunoglobulin-T-cell receptor chimeric molecules as functional receptors with antibody-type specificity. Proc. Natl. Acad. Sci. USA, 86:10024-8.

Gundersen R W, Barrett J N (1979) Neuronal chemotaxis: chick dorsal-root axons turn toward high concentrations of nerve growth factor. Science 206:1079-1080.

Gyuris J, Golemis E, Chertkov H, Brent R (1993) Cdil, a human G1 and S phase protein phosphatase that associates with Cdk2. Cell 75:791-803.

Harteneck C, Plant T D, Schultz G (2000) From worm to man: three subfamilies of TRP channels. Trends Neurosci 23:159-166.

Hempstead B L, Rabin S J, Kaplan L, Reid S, Parada L F, Kaplan D R (1992) Overexpression of the trk tyrosine kinase rapidly accelerates nerve growth factor-induced differentiation. Neuron 9:883-896.

Holland S J, Gale N W, Gish G D, Roth R A, Songyang Z, Cantley L C, Henkemeyer M, Yancopoulos G D, Pawson T (1997) Juxtamembrane tyrosine residues couple the Eph family receptor EphB2/Nuk to specific SH2 domain proteins in neuronal cells. Embo J 16:3877-3888.

Holland S J, Peles E, Pawson T, Schlessinger J (1998) Cell-contact-dependent signalling in axon growth and guidance: Eph receptor tyrosine kinases and receptor protein tyrosine phosphatase beta. Curr Opin Neurobiol 8:117-127.

Huber L J, Chao M V (1995) A potential interaction of p75 and trkA NGF receptors revealed by affinity crosslinking and immunoprecipitation. J Neurosci Res 40:557-563.

Jia M, Li M, Liu X W, Jiang H et al. Voltage sensitive calcium currents are acutely increased by nerve growth factor in PC12 cells. J Neurophysiol 82, 2847 (1999)

Kafitz K W, Rose C R, Thoenen H, Konnerth A (1999) Neurotrophin-evoked rapid excitation through TrkB receptors [see comments]. Nature 401:918-921.

Kang H and Schuman E M. Long-lasting neurotrophin-induced enhancement of synaptic transmission in the adult hippocampus. Science 267, 1658 (1995)

Kaplan D R, Miller F D (2000) Neurotrophin signal transduction in the nervous system. Curr Opin Neurobiol 10:381-391.

Khursigara G, Orlinick J R, Chao M V (1999) Association of the p75 neurotrophin receptor with TRAF6. J Biol Chem 274:2597-2600.

Koizumi S, Contreras M L, Matsuda Y, Hama T, Lazarovici P, Guroff G (1988) K-252a: a specific inhibitor of the action of nerve growth factor on PC 12 cells. J Neurosci 8:715-721.

Korte M, Carroll P, Wolf E, Brem G et al. Hippocampal long-term potentiation is impaired in mice lacking brain-derived neurotrophic factor. Proc Natl Acad Sci 92, 8856 (1995).

Lai C, Lemke G (1991) An extended family of protein-tyrosine kinase genes differentially expressed in the vertebrate nervous system. Neuron 6:691-704.

Letourneau P C (1978) Chemotactic response of nerve fiber elongation to nerve growth factor. Dev Biol 66:183-196.

Levi-Montalcini R (1987) The nerve growth factor 35 years later. Science 237:1154-1162.

Lewin G R, Barde Y A (1996) Physiology of the neurotrophins. Annu Rev Neurosci 19:289-317.

McAllister A K, Katz L C, Lo D C (1999) Neurotrophins and synaptic plasticity. Annu Rev Neurosci 22:295-318.

Meakin S O, MacDonald J I, Gryz E A, Kubu C J, Verdi J M (1999) The signaling adapter FRS-2 competes with Shc for binding to the nerve growth factor receptor TrkA. A model for discriminating proliferation and differentiation. J Biol Chem 274:9861-9870.

Meinkoth et al, “Hybridization of nucleic acids immobilized on solid supports”, Anal Biochem 138:267-284 (1984)

Mellitzer G, Xu Q, Wilkinson D G (2000) Control of cell behaviour by signalling through Eph receptors and ephrins. Curr Opin Neurobiol 10:400-408.

Miller K G, Emerson M D, McManus J R, and Rand J B. (2000) RIC-8 (Synembryn): a novel conserved protein that is required for G_{q}α signaling in the C. elegans nervous system Neuron 27, 289-299.

Miranda J D, White L A, Marcillo A E, Willson C A, Jagid J, Whittemore S R (1999) Induction of Eph B3 after spinal cord injury. Exp Neurol 156:218-222.

Otsuka A J, Franco R, Yang B, Shim K H, Tang L Z, Zhang Y Y, Boontrakulpoontawee P, Jeyaprakash A, Hedgecock E, Wheaton V I, et al. (1995) An ankyrin-related gene (unc-44) is necessary for proper axonal guidance in Caenorhabditis elegans. J Cell Biol 129:1081-1092.

Patel T D, Jackman A, Rice F L, Kucera J, Snider W D (2000) Development of sensory neurons in the absence of NGF/TrkA signaling in vivo [see comments]. Neuron 25:345-357.

Paxinos G (1995) The Rat Nervous System, 2nd Edition. San Diego: Academic Press, Inc.

Pinon L G, Minichiello L, Klein R, Davies A M (1996) Timing of neuronal death in trkA, trkB and trkC mutant embryos reveals developmental changes in sensory neuron dependence on Trk signalling. Development 122:3255-3261.

Poo, Neurotrophins as synaptic modulators. Nature Rev Neurosci 2:24-31 (2001)

Pozzo-Miller L D, Gottschalk W, Zhang L et al. Impairments in high-frequency transmission, synaptic vesicle docking and synaptic protein distribution in the hippocampus of BDNF knockout mice. J Neuroscience 19, 4972 (1999)

Qian X, Riccio A, Zhang Y, Ginty D D (1998) Identification and characterization of novel substrates of Trk receptors in developing neurons. Neuron 21:1017-1029.

Ross A H, Daou M C, McKinnon C A, Condon P J, Lachyankar M B, Stephens R M, Kaplan D R, Wolf D E (1996) The neurotrophin receptor, gp75, forms a complex with the receptor tyrosine kinase TrkA. J Cell Biol 132:945-953.

Sala R, Viegi A, Rossi F M et al. Nerve growth factor and brain-derived neurotrophic factor increase neurotransmitter release in the rat visual cortex. Eur J Neurosci 10, 2185 (1998)

Sheng M & Sala C, PDZ domains and the organization of supramolecular complexes, Ann Rev Neurosci 24, 1-29, (2001)

Smith C A, Farrah T, Goodwin R G (1994) The TNF receptor superfamily of cellular and viral proteins: activation, costimulation, and death. Cell 76:959-962.

Snider W D (1994) Functions of the neurotrophins during nervous system development: what the knockouts are teaching us. Cell 77:627-638.

Srivastava S, Osten P, Vilim F S, Khatri L, Inman G, States B, Daly C, DeSouza S, Abagyan R, Valtschanoff J G, Weinberg R J, and Ziff E B (1998) Novel anchorage of GluR2/3 to the postsynaptic density by the AMPA receptor-binding protein ABP. Neuron 21, 581-591.

Thoenen H (1995) Neurotrophins and neuronal plasticity. Science 270:593-598.

Toledo-Aral J J, Brehm P, Halegoua S and Mandel G. A single pulse of nerve growth factor triggers long-term neuronal excitability through sodium channel gene induction. Neuron 14, 607 (1995).

Torres R, Firestein B L, Dong H, Staudinger J, Olson E N, Huganir R L, Bredt D S, Gale N W, Yancopoulos G D (1998) PDZ proteins bind, cluster, and synaptically colocalize with Eph receptors and their ephrin ligands [see comments]. Neuron 21:1453-1463.

Verdi J M, Birren S J, Ibanez C F, Persson H, Kaplan D R, Benedetti M, Chao M V, Anderson D J (1994) p75LNGFR regulates Trk signal transduction and NGF-induced neuronal differentiation in MAH cells. Neuron 12:733-745.

Welch M D, DePace A H, Verma S, Iwamatsu A, and Mitchison T J. (1997) The human Arp2/3 complex is composed of evolutionarily conserved subunits and is localized to cellular regions of dynamic actin filament assembly. J Cell Biol 138, 375-384.

Wright D E, Snider W D (1995) Neurotrophin receptor mRNA expression defines distinct populations of neurons in rat dorsal root ganglia. J Comp Neurol 351:329-338.

Yan Q, Johnson E M, Jr. (1988) An immunohistochemical study of the nerve growth factor receptor in developing rats. J Neurosci 8:3481-3498.

Yan Q, Radeke M J, Matheson C R, Talvenheimo J, Welcher A A, Feinstein S C (1997) Immunocytochemical localization of TrkB in the central nervous system of the adult rat [published erratum appears in J Comp Neurol 1997 June 16;382(4):546-7]. J Comp Neurol 378:135-157.

What is claimed is:

1. An isolated polypeptide, which associates with TrkA and p75 neurotrophin receptors, is a target for phosphorylation by neurotrophin and ephrin receptor tyrosine kinases, enhances neurotransmitter release, and modulates the clustering of proteins involved in ion channel formation, comprising the amino acid sequence of:
(A) SEQ ID NO:2;
(B) SEQ ID NO:4;
(C) a fragment of the polypeptide of SEQ ID NO:2;
(D) a fragment of the polypeptide of SEQ ID NO:4;
(E) a variant polypeptide which is at least 95% identical to SEQ ID NO:2;
(F) a variant polypeptide which is at least 95% identical to SEQ ID NO:4; or
(G) a functional derivative or a salt of (A), (B), (C), (D), (E), or (F)
wherein said fragments (C) and (D) and said variants (E) and (F) have the properties of associating with TrkA and p75 neurotrophin receptors, being a target for phosphorylation by neurotrophin and ephrin receptor tyrosine kinase, enhancing neurotransmitter release, and modulating the clustering of proteins involved in ion channel formation.

2. The polypeptide of claim 1, which comprises the amino acid sequence of SEQ ID NO:2.

3. The polypeptide of claim 1, which comprises the amino acid sequence of SEQ ID NO:4.

4. The polypeptide of claim 1, which comprises the amino acid sequence of fragment (C).

5. The polypeptide of claim 4, wherein said fragment (C) either further contains one or more transmembrane domains of the polypeptide of SEQ ID NO:2 or is fused to a transmembrane domain to form a fusion polypeptide.

6. The polypeptide of claim 1, which comprises the amino acid sequence of fragment (D).

7. The polypeptide of claim 6, wherein said fragment (D) either further contains one or more transmembrane domain of the polypeptide of SEQ ID NO:4 or is fused to a transmembrane domain to form a fusion polypeptide.

8. The polypeptide of claim 1, which comprises the amino acid sequence of a variant polypeptide which is at least 95% identical to SEQ ID NO:2.

9. The polypeptide of claim 1, which comprises the amino acid sequence of a variant polypeptide which is at least 95% identical to SEQ ID NO:4.

10. A molecule which contains the antigen-binding portion of an antibody specific for the polypeptide of claim 1.

11. The molecule of claim 10, which is a monoclonal antibody.

12. The molecule of claim 10, which is a single chain antibody.

13. The molecule of claim 10, which is specific for a polypeptide of SEQ ID NO:2.

14. A method for visualizing the growth cone of neurons, comprising:
contacting the molecule of claim 13 with neurons to detect the presence of the polypeptide of SEQ ID NO:2 as a marker for the growth cone of neurons; and
visualizing the growth cone of neurons by the binding of the molecule of claim 13 to the polypeptide of SEQ ID NO:2 localized in the growth cone of neurons.

15. The molecule of claim 10, which is specific for a polypeptide of SEQ ID NO:4.

16. A method for visualizing the growth cone of neurons, comprising:
contacting the molecule of claim 15 with neurons to detect the presence of the polypeptide of SEQ ID NO:4 as a marker for the growth cone of neurons; and
visualizing the growth cone of neurons by the binding of the molecule of claim 15 to the polypeptide of SEQ ID NO:4 localized in the growth cone of neurons.

17. An isolated nucleic acid encoding the polypeptide of claim 1.

18. The nucleic acid of claim 17, which encodes a polypeptide comprising SEQ ID NO:2.

19. The nucleic acid of claim 18, comprising a nucleotide sequence of SEQ ID NO:1.

20. The nucleic acid of claim 17, which encodes a polypeptide comprising SEQ ID NO:4.

21. The nucleic acid of claim 20, comprising a nucleotide sequence of SEQ ID NO:3.

22. The nucleic acid of claim 17, which encodes the amino acid sequence of fragment (C).

23. The nucleic acid of claim 17, which encodes the amino acid sequence of fragment (D).

24. The nucleic acid of claim 17, which encodes a variant polypeptide which is at least 95% identical to SEQ ID NO:2.

25. The nucleic acid of claim 17, which encodes a variant polypeptide which is at least 95% identical to SEQ ID NO:4.

26. A vector comprising the nucleic acid of claim 17.

27. A host cell transformed with the nucleic acid of claim 17.

28. A method for producing a polypeptide which associates with TrkA and p75 neurotrophin receptors, is a target for phosphorylation by neurotrophin and ephrin receptor tyrosine kinases, enhances neurotransmitter release, and modulates the clustering of proteins involved in ion channel formation, comprising:
culturing the host cell of claim 27 in a nutrient medium to express and produce the polypeptide; and
recovering the produced polypeptide.

29. An isolated nucleic acid, which specifically hybridizes under stringent conditions to the complement of either SEQ ID NO:1 or SEQ ID NO:3.

30. The nucleic acid of claim 29, which specifically hybridizes under highly stringent conditions to the complement of SEQ ID NO:1.

31. The nucleic acid of claim 29, which specifically hybridizes under hybridizes under highly stringent conditions to the complement of SEQ ID NO:3.

32. A vector comprising the nucleic acid of claim 29.

33. A host cell transformed with the nucleic acid of claim 29.

34. A method for producing a polypeptide which associates with TrkA and p75 neurotrophin receptors, is a target for phosphorylation by neurotrophin and ephrin receptor tyrosine kinases enhances neurotransmitter release, and modulates the clustering of proteins involved in ion channel formation, comprising:
culturing the host cell of claim 33 in a nutrient medium to express and produce the polypeptide; and
recovering the produced polypeptide.
